(19) United States
(12) Patent Application Publication
(10) Pub. No.: US 2005/0176076 A1
(43) Pub. Date: Aug. 11, 2005

(54) Multiplex protease profiling

(75) Inventors: Hongye Sun, San Mateo, CA (US); Lawrence N. Greenfield, Eugene, OR (US); Douglas A. Bost, San Mateo, CA (US)

Correspondence Address: JONES DAY, 222 EAST 41ST ST, NEW YORK, NY, 10017, US

(73) Assignee: Applera Corporation

(21) Appl. No.: 11/025,608

(22) Filed: Dec. 28, 2004

Related U.S. Application Data
(60) Provisional application No. 60/533,865, filed on Dec. 31, 2003.

(51) Int. Cl. G01N 33/574; G01N 33/53; G01N 33/537; G01N 33/543; C12Q 1/37

(52) U.S. Cl. 435/7.23; 435/7.92; 435/23

(57) ABSTRACT

In certain embodiments, provided are methods and compositions useful for the detection and quantitation of catalytically active enzymes, particularly catalytically active proteases. The compositions comprise one or more multifunctional tags, wherein each different multifunctional tag comprises a different mobility modifier, a partitioner, a reporter, and a peptide substrate that is specifically or substantially specifically hydrolyzed by a target protease or target protease family. Hydrolysis of each different multifunctional tag by a target protease provides a different labeled hydrolytic product comprising a reporter and a mobility modifier but not a partitioner, where the mobility modifier confers a distinctive mobility on the labeled hydrolytic product in a mobility-dependent analysis technique.

The invention relates to compositions and methods useful for the detection and quantitation of catalytically active enzymes.

### 1. SUMMARY

In some embodiments, the present invention is directed toward compositions and methods useful for the multiplex analysis of catalytically-active hydrolases, particularly proteases.

In some embodiments, there are provided methods for detecting the presence or absence of a plurality of proteases in a sample. In practicing such methods, a plurality of different multifunctional tags are added to a sample comprising a plurality of target proteases under conditions conducive for hydrolysis of a multifunctional tag by a specific protease to provide at least two products. One, or at least one, of the products of each hydrolysis reaction comprises a reporter and a mobility modifier that imparts a distinctive electrophoretic mobility to that product.

In some embodiments, there are provided compositions comprising multifunctional tags that can be used to detect the presence or absence of one or more catalytically-active target proteases in a sample. The multifunctional tag composition comprises a plurality of different multifunctional tags, each of which comprises a peptide substrate that is substantially specifically hydrolyzable by a different catalytically-active target protease, a distinctive mobility modifier, a partitioner, and a reporter. Hydrolysis of the peptide substrate of each different multifunctional tag by a different catalytically-active target protease present in a sample provides a different labeled hydrolytic product that comprises a reporter and a distinctive mobility modifier but does not include the partitioner. Each distinctive mobility modifier imparts to each different labeled hydrolytic product an electrophoretic mobility that is distinctive relative to the electrophoretic mobility of each other different multifunctional tag in the composition and is also distinctive relative to other labeled hydrolytic products produced by hydrolysis of the peptide substrate of other different multifunctional tags by other different catalytically-active target proteases.

In some embodiments, a reporter, which can be or comprise, without limitation, a fluorescent dye, is attached to either or both of the peptide substrate or the mobility modifier. In another aspect of this embodiment, the peptide substrate comprises fewer than 50, fewer than 40, fewer than 30, fewer than 20 or fewer than 15 amino acids.

In some embodiments, the mobility modifier of each multifunctional tag is a substantially monodisperse polymer which can be, in various non-limiting examples, a polyethylene oxide, polyglycolic acid, polylactic acid, oligosaccharide, polyurethane, polyamide, polyamine, polyimine, polysulfonamide, polysulfoxide, or a block copolymer thereof. In some embodiments, the multifunctional tag comprises a polyethylene oxide polymer, which can include a charged linking group, such as a phosphodiester linking group, or an uncharged linking group, such as a phosphotriester linking group.

In some embodiments, the partitioner can be solid surface or can be polymer. Such polymers include, but are not limited to polyethylene oxide, polyglycolic acid, polylactic acid, oligosaccharide, polyurethane, polyamide, polyamine, polyimine, polysulfonamide, polysulfoxide, and block copolymers thereof. Where the partitioner is a polymer it can, but need not, be a substantially monodisperse polymer. In one illustrative example, the partitioner is a polyethylene oxide polymer, which can include a charged linking group, such as but not limited to a phosphodiester linking group, and/or can include an uncharged linking group, such as but not limited to a phosphotriester linking group.

In some embodiments, a multifunctional tag has a net negative electrostatic charge and comprises a partitioner carrying a net negative electrostatic charge. However, the labeled hydrolytic product generated by hydrolysis of the peptide substrate of this negatively-charged multifunctional tag provides a labeled hydrolytic product, which includes a mobility-modifier and a reporter but not a partitioner, that carries a net positive electrostatic charge. In some embodiments, the multifunctional tag carries a net positive electrostatic charge and comprise a partitioner carrying a net positive electrostatic charge, while the labeled hydrolytic product generated therefrom carries a net negative electrostatic charge. In some embodiments, each multifunctional tag of a composition comprises a partitioner has a molecular weight that at least twice, at least five times, or at least ten times greater than the molecular weight of any labeled hydrolytic product generated by hydrolysis of the multifunctional tags of the composition.

Also provided are methods for detecting the presence or absence of one or more catalytically-active target enzymes, particularly proteases, in a sample. Such methods involve contacting the sample with a multifunctional tag composition under selected hydrolysis conditions to provide a reaction mixture. The multifunctional tag composition comprises a plurality of different multifunctional tags, each of which includes a peptide substrate that is substantially specifically hydrolyzed by a different catalytically-active protease, a distinctive mobility modifier and partitioner attached to each peptide substrate, and a reporter.

Hydrolysis of each different multifunctional tag by a different target protease provides a different labeled hydrolytic product, each of which comprises a distinctive mobility modifier and a reporter but does not include a partitioner. The mobility modifier imparts to each different labeled hydrolytic product a distinctive electrophoretic mobility relative to the electrophoretic mobility of the other different multifunctional tags in the composition and relative to the electrophoretic mobility of other different labeled hydrolytic products in the reaction mixture. The reaction mixture, which contains the labeled hydrolytic products, is fractionated using a mobility-dependent analysis technique and one or more different labeled hydrolytic products are then detected. Since each different labeled hydrolytic product is generated by hydrolysis of a different multifunctional tag by a specific catalytically-active target protease, the presence of each different labeled hydrolytic product indicates that a different catalytically-active target protease is present in the sample. Similarly, the absence of each different labeled hydrolytic product can indicate that a different catalytically-active target protease is absent from the sample. The amount of each different labeled hydrolytic product may be substantially proportional to the amount of each different catalytically-active target protease present in the sample. In some embodiments, fractionation is carried out using electrophoresis. In some embodiments, the electrophoresis is capillary electrophoresis, which can be conducted in a sieving medium or in a non-sieving medium. In some embodiments, an electrophoretic separation is carried out in the presence of an affinophore comprising a first ligand, where at least one multifunctional tag of the composition comprises a mobility modifier comprising a second ligand, where the first ligand and the second ligand are members of a binding pair.

Also provided are kits for detecting one or more catalytically-active target enzymes, particularly proteases in a sample. The kit comprises a plurality of different multifunctional tags. Each different multifunctional tag of such a kit comprises a peptide substrate that is substantially specifically hydrolyzed by a different catalytically-active target protease, a distinctive mobility modifier and partitioner attached to the peptide substrate, and a reporter. Hydrolysis of the peptide substrate of a different multifunctional tag by a different catalytically-active target protease provides a different labeled hydrolytic product, which includes a reporter and a distinctive mobility modifier but does not comprise a partitioner. Each distinctive mobility modifier imparts to each different labeled hydrolytic product a distinctive electrophoretic mobility relative to the electrophoretic mobility of the other different multifunctional tags and of other different labeled hydrolytic products provided by hydrolysis of the peptide substrate of other different multifunctional tag by a different catalytically-active target protease.

Also provided are methods for diagnosing a disease in a subject. Such methods comprise providing a sample derived from a tissue of the subject, where that sample comprises at least one catalytically-active target protease, as well as providing a multifunctional tag composition that comprises a plurality of different multifunctional tags. Each different multifunctional tag comprises a peptide substrate substantially specifically hydrolyzed by a different catalytically-active target protease, a distinctive mobility modifier attached to the peptide substrate, partitioner attached to the peptide substrate, and a reporter.

The sample and the multifunctional tag composition are combined under selected hydrolysis conditions to provide a reaction mixture. Under such conditions, hydrolysis of each different multifunctional tag by each different catalytically-active target protease provides a different labeled hydrolytic product, which comprises a distinctive mobility modifier and a reporter but does not comprise a partitioner. The mobility modifier imparts to each different labeled hydrolytic product a distinctive electrophoretic mobility relative to the electrophoretic mobility of the other different multifunctional tags in the reaction and of other different labeled hydrolytic products in the reaction. According to this method, a first labeled hydrolytic product is diagnostic of normal tissue and a second labeled hydrolytic product is diagnostic of diseased tissue. The reaction mixture is fractionated using a mobility-dependent analysis technique and each different labeled hydrolytic product is detected. In some embodiments, the electrophoretic separation is carried out carried out in a sieving medium while in other embodiments, the electrophoretic separation is carried out in a non-sieving medium. Where the amount of the first labeled hydrolytic product detected is greater than that of the second labeled hydrolytic product, the method indicates that the tissue is normal. In contrast where the amount of the second labeled hydrolytic product detected is greater than that for the first labeled hydrolytic product, the method indicates that the tissue is diseased. Diseased tissue examined can be a tissue of a type of cancer or it can be tissue infected by an infectious agent such as, but not limited to, a bacterial, fungal, parasitic, or viral infectious agent. In certain, non-limiting examples the infectious agent is an HIV virus or a viral infectious agent that is a causative agent of SARS, (“severe acute respiratory syndrome”).

Also provided are methods of screening for therapeutic agents useful for the prevention and treatment of disease. Such methods comprises providing a sample comprising a plurality of different catalytically-active target proteases, each of which is diagnostic of a different target disease. The method also involves providing two multifunctional tag compositions. The first multifunctional tag composition comprises a first set of first different multifunctional tags, wherein each first multifunctional tag comprises a first peptide substrate substantially specifically hydrolyzable by a different catalytically-active target protease, a first distinctive mobility modifier attached to the first peptide substrate, a first partitioner attached to the first peptide substrate, and a first reporter. The second multifunctional tag composition comprises a test compound and a second set of second different multifunctional tags, wherein each second different multifunctional tag comprises a second peptide substrate substantially specifically hydrolyzable by a different target protease, a second distinctive mobility modifier attached to the second peptide substrate, a second partitioner attached to the second peptide substrate, and a second reporter.

An aliquot of the sample and the first multifunctional tag composition are contacted under selected hydrolysis conditions to produce a first reaction mixture and to provide a first set of first different labeled hydrolytic products. Each first different labeled hydrolytic product comprises a first distinctive mobility modifier and a first reporter but not a first partitioner Each first different labeled hydrolytic product has an electrophoretic mobility that is distinctive relative to the electrophoretic mobility of the first and second different multifunctional tags and relative to the electrophoretic mobility of other first different labeled hydrolytic products in the first reaction mixture. Such differences in electrophoretic mobility can, but need not be, the result of distinctive ratios of charge to translational frictional drag. The amount of each first different labeled hydrolytic product is proportional to the total catalytic activity of a different catalytically-active target protease in the absence of a test compound.

Such methods may also involve contacting an aliquot of the sample and the second multifunctional tag composition under selected hydrolysis conditions to provide a second reaction mixture and to provide a second set of second different labeled hydrolytic products. Each second different labeled hydrolytic product comprises a second distinctive mobility modifier and a second reporter but not a second partitioner. Each second different labeled hydrolytic product has an electrophoretic mobility that is distinctive relative to the electrophoretic mobility of the first and second different multifunctional tags and is distinctive relative to electrophoretic mobility of the first different labeled hydrolytic products and other second different labeled hydrolytic products in the second reaction mixture. Such differences in electrophoretic mobility can, but need not be, the result of distinctive ratios of charge to translational frictional drag. Moreover, the amount of each second labeled hydrolytic product may be proportional to the total catalytic activity of a different catalytically-active target protease in the presence of the test compound.

In some embodiments, the first and second reaction mixtures are combined to provide a combined reaction mixture that is fractionated using a mobility-dependent analysis technique and each first different labeled hydrolytic product and each second different labeled hydrolytic product are detected. The amount of first different labeled hydrolytic product provided by hydrolysis of the peptide substrate of a first different multifunctional tag by a specific catalytically-active target protease and the amount of second different labeled hydrolytic product provided by hydrolysis of the peptide substrate of a second different multifunctional tag by the specific catalytically-active target protease are then determined to evaluate whether or not the test compound inhibited the activity of that specific target protease. In one aspect of this embodiment, each first partitioner and each second partitioner are the same. In another aspect of this embodiment, a first peptide substrate and a second peptide substrate are the same.

In some embodiments, a first different multifunctional tag comprises a first peptide substrate, a first mobility modifier and a first reporter, and a second different multifunctional tag comprises a second peptide substrate, a second mobility modifier and a second reporter, wherein the first peptide substrate and the second peptide substrate are the same, and wherein the first mobility modifier and the second mobility modifier are the same. However, in this aspect the first and second reporters are different, spectrally-resolvable fluorescent dyes. In some embodiments, a first different multifunctional tag comprises a first peptide substrate, a first mobility modifier and a first reporter, and a second different multifunctional tag comprises a second peptide substrate, a second mobility modifier and a second reporter where the first and second peptide substrates are the same and the first and second reporters are the same. However, hydrolysis of the first different multifunctional tags by a target protease provides a first labeled hydrolytic product comprising a first mobility modifier, while hydrolysis of the second different multifunctional tag by the target protease provides a second different hydrolytic product comprising the second mobility modifier. In this aspect, the first mobility modifier imparts distinctive electrophoretic mobility to the first labeled hydrolytic product that is distinctive relative to the electrophoretic mobility imparted by the second mobility modifier to the second different labeled hydrolytic product. This difference in electrophoretic mobility can, but need not be, the result of a distinctive ratio of charge to translational frictional drag.

### 2. BRIEF DESCRIPTION OF THE DRAWINGS

FIGS. 1A-1B illustrate two general types of multifunctional tags in which the partitioner employed to facilitate separation of the labeled hydrolytic product from nonhydrolyzed multifunctional tags is either a relatively high molecular weight polymer (FIG. 1A) or a solid surface (FIG. 1B).

FIGS. 2A-2B generally illustrate multifunctional tags in which the relative electrostatic charge of the multifunctional tag and the partitioner differ from that of the labeled hydrolytic product.

### 3. DETAILED DESCRIPTION OF SOME EMBODIMENTS

Reference will now be made in detail to some embodiments of the present invention, examples of which are illustrated in the accompanying drawings. While the invention will be described in conjunction with these embodiments, it will be understood that the invention is not intended to be limited to those embodiments. On the contrary, the invention is intended to cover alternatives, modifications, and equivalents of these preferred embodiments that may be included within the invention as defined by the appended claims.

Proteases regulate many different cell proliferation, differentiation, and signaling processes by regulating protein turnover and processing. It has been asserted that proteases are involved in the regulation of most physiological processes, playing a central role in apoptosis, protein hormone processing, the complement system, fibrinolysis, and blood coagulation (J. A. Ellman (2000) Chapter 6, “Combinatorial Methods to Engineer Small Molecules for Functional Genomics,” Ernst Schering Research Foundation Workshop, 32: 183-204). Proteases are ubiquitous throughout nature and have been estimated to make up approximately 2% of all gene products, suggesting that the human genome encodes approximately 700 proteases.

Proper functioning of the cell requires precise control of the levels of critical structural proteins, enzymes, and regulatory proteins. One of the ways that cells can reduce the steady state level of a particular protein is by proteolytic degradation. Thus, complex and highly-regulated mechanisms have been evolved to accomplish this degradation. Uncontrolled protease activity (either increased or decreased) has been implicated in a variety of disease conditions including inflammation, cancer, arteriosclerosis, and degenerative disorders.

One example of a specific protease believed to be involved in the spread of cancer is the extracellular proteases hepsin. This protease mediates the digestion of neighboring extracellular matrix components in initial tumor growth, allow shedding or desquamation of tumor cells into the surrounding environment, provide the basis for invasion of basement membranes in target metastatic organs, and are required for release and activation of many growth and angiogenic factors. Experimental evidence indicates that hepsin, a cell surface serine protease identified in hepatoma cells, is overexpressed in ovarian cancer. Hepsin does not appear to be essential for development or homeostasis. On Northern blot analysis, the hepsin transcript was abundant in carcinoma but was almost never expressed in normal adult tissue, including normal ovary, suggesting that hepsin is frequently overexpressed in ovarian tumors and therefore may be a candidate protease diagnostic for the invasive process and growth capacity of ovarian tumor cells (see Tanimoto et al., (1997) Cancer Res. 57(14): 2884-7; Leytus et al. (1988) Biochemistry 27: 1067-1074; Tsuji et al. (1991) J. Biol. Chem. 266: 16948-16953; and Wu et al. (1998) J. Clin. Invest. 101: 321-326).

Physiologically important proteases include, but are not limited to, members of the metalloprotease, serine protease, cysteine protease, and aspartic protease families.

Metalloproteases contain a catalytic zinc metal center which participates in the hydrolysis of the peptide backbone (reviewed in Power and Harper, in Protease Inhibitors, A. J. Barrett and G. Salversen (eds.) Elsevier, Amsterdam, 1986, p. 219). The active zinc center differentiates some of these proteases from calpains and trypsins whose activities are dependent upon the presence of calcium. Examples of metalloproteases include carboxypeptidase A, thermolysin, membrane alanyl aminopeptidase, germinal peptidyl-dipeptidase A, collagenase 1, neprilysin, membrane dipeptidase, and S2P protease. Metalloproteases are believed to have a number of roles in vivo including proteolytic processing of the vasoconstrictor, endothelin-1, and processing of peptide hormones.

A number of diseases are thought to be mediated by excess or undesired metalloprotease activity or by an imbalance in the relative activity of one or more member of the protease family of proteins. These include: (a) osteoarthritis (Woessner et al. (1984) J. Biol. Chem. 259(6): 3633; Phadke et al. (1983) J. Rheumatol. 10: 852); (b) rheumatoid arthritis (Mullins et al. (1983) Biochim. Biophys. Acta 695: 117; Woolley et al. (1977) Arthritis Rheum. 20: 1231; Gravallese, et al. (1991) Arthritis Rheum. 34: 1076); (c) septic arthritis (Williams et al. (1990) Arthritis Rheum. 33: 533); (d) tumor metastasis (Reich et al. (1988) Cancer Res. 48: 3307, and Matrisian et al. (1986) Proc. Natl. Acad. Sci., USA 83: 9413); (e) periodontal diseases (Overall et al. (1987) J. Periodontal Res. 22: 81); (f) corneal ulceration (Burns et al. (1989) Invest. Opthalmol. Vis. Sci. 30: 1569); (g) proteinuria (Baricos et al. (1988) Biochem. J. 254: 609); (h) coronary thrombosis from atherosclerotic plaque rupture (Henney et al. (1991) Proc. Natl. Acad. Sci. USA 88: 8154-8158); (i) aneurysmal aortic disease (Vine et al. (1991) Clin. Sci. 81: 233); (j) birth control (Woessner et al. (1989) Steroids 54: 491); (k) dystrophobic epidermolysis bullosa (Kronberger et al. (1982) J. Invest. Dermatol. 79: 208); (1) degenerative cartilage loss following traumatic joint injury; (m) conditions leading to inflammatory responses, osteopenias mediated by matrix metalloprotease activity; (n) tempero-mandibular joint disease; and (O) demyelinating diseases of the nervous system (Chantry et al. (1988) J. Neurochem. 50: 688).

The matrix metalloproteinases, a subfamily of the metalloproteinases, include at least 19 zinc-dependent proteases roughly grouped into four classes: gelatinases, stromelysins, membrane-type matrix metalloproteinases, and collagenases. Physiologically, the matrix metalloproteinases are involved in normal remodelling of tissues during wound healing, ovulation, angiogenesis, mammary gland involution, and embryonic development. Abnormal expression of matrix metalloproteinases is believed to be contribute to pathological conditions including tumor growth, invasiveness, and metastasis, pulmonary emphysema, rheumatoid arthritis, and osteoarthritis. Moreover, increased levels of the matrix metalloproteinases MMP-2 and stromelysin-3 have been detected in certain breast cancers and have been correlated with a poor prognosis (see Duffy et al. (2000) Breast Cancer Res. 2: 252-57 and references cited therein).

Proteases are critical elements at several stages in the progression of metastatic cancer. In this process, the proteolytic degradation of structural protein in the basal membrane allows for expansion of a tumor in the primary site, escape from this site and metastasis to noncontiguous secondary sites. In addition, angiogenesis, which is required for tumor growth and survival, is dependent on proteolytic tissue remodeling. Transfection experiments with various types of proteases have shown that the matrix metalloproteases, e.g. gelatinases A and B (MMP-2 and MMP-9, respectively), play a dominant role in these processes (see Mullins et al. (1983) Biochim. Biophys. Acta 695: 177; Ray et al. (1994) Eur. Respir. J. 7: 2062; and Birkedal-Hansen et al. (1993) Crit. Rev. Oral Biol. Med. 4: 197).

At least 11 human caspases (cysteine aspartate proteases) have been identified. Caspases have been shown to play a central role at different stages of apoptosis (programmed cell death). In addition to the caspases, it has also been shown that members other protease families, e.g. the calpains, serine proteinases, and metalloproteinases, also play a role in apoptosis (see Grttüer M G (2000) Curr Opin Struct Biol. 10(6): 649-55; and Mykles, D. L. (2001) Methods Cell Biol. 66: 247-87). Accordingly, it is important to be able to determine level of such enzymatically active proteases in tissue samples e.g., since insufficient apoptosis is associated with pathological conditions including cancer and autoimmune disease, while excessive apoptosis is associated with neurodegenerative conditions and ischemic damage to tissues.

The serine proteases are a large family of proteolytic enzymes that include the digestive enzymes, trypsin and chymotrypsin, components of the complement cascade and of the blood-clotting cascade, and enzymes that control the degradation and turnover of macromolecules of the extracellular matrix. Serine proteases are so named because of the presence of a serine residue in the active catalytic site for protein hydrolysis. Serine proteases have a wide range of substrate specificities and can be subdivided into subfamilies on the basis of these specificities. The main sub-families are trypases (hydrolysis after arginine or lysine), aspases (hydrolysis after aspartate), chymases (hydrolysis after phenylalanine or leucine), metases (hydrolysis after methionine), and serases (hydrolysis after serine). A series of six serine proteases have been identified in murine cytotoxic T-lymphocytes (CTL) and natural killer (NK) cells. These serine proteases are involved with CTL and NK cells in the destruction of virally transformed cells and tumor cells and in organ and tissue transplant rejection (Zunino et al. (1990) J. Immunol. 144: 2001-9; and Sayers et al. (1994) J. Immunol. 152: 2289-97). Human homologs of most of these enzymes have been identified (Trapani et al. (1988) Proc. Natl. Acad. Sci. USA 85: 6924-28; Caputo et al. (1990) J. Immunol. 145: 737-44).

The serine proteases are secretory proteins which contain N-terminal signal peptides that serve to export the immature, catalytically-inactive protein across the endoplasmic reticulum and are then cleaved (von Heijne (1986) Nuc. Acid. Res. 14: 5683-90). Serine proteases, particularly the digestive enzymes, exist as inactive precursors or preproenzymes, and contain a leader or activation peptide sequence 3′ of the signal peptide. This activation peptide may be 2-12 amino acids in length, and it extends from the hydrolysis site of the signal peptide to the N-terminal IIGG sequence of the active, mature protein. Hydrolysis of this sequence activates the enzyme. This sequence varies in different serine proteases according to the biochemical pathway, the substrate (Zunino et al., supra; Sayers et al., supra) and the sequence of a substrate binding sites which are believed to determine serine protease substrate specificities (Zunino et al. (1990) J. Immunol. 144: 2001-09).

The trypsinogens are serine proteases secreted by exocrine cells of the pancreas (Travis et al. (1969) Biochemistry 8: 2884-89; and Mallory et al. (1973) Biochemistry 12: 2847-51). Two major types of trypsinogen isoenzymes have been characterized, trypsinogen-1, also called cationic trypsinogen, and trypsinogen-2 or anionic trypsinogen. The trypsinogen proenzymes are activated to trypsins in the intestine by enterokinase, which removes an activation peptide from the N-terminus of the trypsinogens. The trypsinogens show a high degree of sequence homology, but they can be separated on the basis of charge differences by using electrophoresis or ion exchange chromatography. Although the major form of trypsinogen in the pancreas, pancreatic juice, and the serum of healthy individuals, is trypsinogen-1 (Guy et al. (1984) Biochem Biophys Res Commun 125: 516-23). However, trypsinogen-2 is more strongly elevated in the serum of patients afflicted with pancreatitis (Itkonen et al. (1990) J Lab Clin Med 115: 712-18). Trypsinogens also occur in certain ovarian tumors, in which trypsinogen-2 is the major form (Koivunen et al. (1990) Cancer Res 50: 2375-78). In one theory, acute pancreatitis is caused by autodigestion resulting from premature activation of proteolytic enzymes in the pancreas rather than in the duodenum. Any number of other factors including endotoxins, exotoxins, viral infections, ischemia, anoxia, and direct trauma may activate the proenzymes.

Most of aspartic proteases belong to the pepsin family. The pepsin family includes digestive enzymes such as pepsin and chymosin as well as lysosomal cathepsins D and processing enzymes such as renin. Examples of the aspartic protease family of proteins include, but are not limited to, pepsin A (Homo sapiens), HIV1 retropepsin (human immunodeficiency virus type 1), polyprotein peptidase (human spumaretrovirus), and presenilin 1 (Homo sapiens).

3.1 Definitions

Unless stated otherwise, the following terms and phrases used herein are intended to have the following meanings.

As used herein, the phrase “substantially protease specific peptide substrate,” refers to a peptide that is hydrolyzable by a particular protease or protease family with a hydrolytic efficiency that is at least twice that of any other protease or protease family.

As used herein, the phrase “substantially protease-specific multifunctional tag,” refers to a multifunctional tag comprising a peptide that is hydrolyzed by a particular protease or protease family with a hydrolytic efficiency that is at least twice that of any other protease or protease family.

The phrase “protease family,” as used herein encompasses any set or collection of proteins that can be classified together either by virtue of their amino acid sequence similarity or homology, or by virtue of the commonality of substrates cleaved by the proteases. As used herein, the phrase “protease family” may also encompass a group, set or collection of proteases used to provide a “fingerprint” of proteolytic activity characteristic of e.g. normal tissue or of diseased tissue.

As used herein, the term “ligand” refers to a chemical moiety or structure corresponding to one member of a cognate binding pair that is specifically recognized and bound in a stable complex by a second member of the cognate binding pair. Examples of such cognate binding pairs include, but are not limited to, biotin-avidin, and biotin-streptavidin. Other examples include phenyl boronic acid reagents and phenyl boronic acid complexing reagents derived from aminosalicylic acid (see e.g. U.S. Pat. No. 5,594,151, and U.S. Pat. No. 6,414,122 B1,each of which is hereby incorporated by reference in its entirety). Therefore, as used herein, the term ligand encompasses the term hapten, which refers to a chemical moiety or structure, for example digoxigenin, as one member of a cognate binding pair, where the second member of the cognate binding pair is an component of the immune system, including but not limited to an intact antibody, a single chain antibody, or an antibody fragment.

“Linker” refers to a moiety that links one moiety to another, e.g.: (i) a reporter to a mobility modifier or to a peptide substrate or (ii) a peptide substrate to a solid support or surface.

“Linking group” means a moiety capable of reacting with a “complementary functionality” to form a “linkage.” A linking group and its associated complementary functionality is referred to herein as a “linkage pair.” Exemplary linkage pairs include a first member selected from the group isothiocyanate, sulfonyl chloride, 4,6-dichlorotriazinyl, succinimidyl ester, or other active carboxylate, and a second member that is amine, hydroxyl, or sulfhydryl. In some embodiments, a first member of a linkage pair is maleimide, halo acetyl, or iodoacetamide whenever the second member of the linkage pair is sulfhydryl (e.g., R. Haugland, Molecular Probes Handbook of Fluorescent Probes and Research Chemicals, Molecular probes, Inc. (1992)). In some embodiments, the first member of a linkage pair is N-hydroxysuccinimidyl (NHS) ester and the second member of the linkage pair is amine, where, to form an NHS ester, a carboxylate moiety is reacted with dicyclohexylcarbodiimide and N-hydroxysuccinimide.

“Attachment site” refers to a site on a moiety to which a linker or linking group is covalently attached.

“Mobility-dependent analysis technique” means an analysis technique based on differential rates of migration between different analyte species. Exemplary mobility-dependent analysis techniques include electrophoresis, particularly capillary electrophoresis both in sieving and in non-sieving media, chromatography, sedimentation, e.g., gradient centrifugation, field-flow fractionation, multi-stage extraction techniques and the like.

The “translational frictional drag” of a polymer is a measure of the polymer's frictional drag as it moves electrophoretically through a defined, non-sieving liquid medium.

A “distinctive ratio of charge/translational frictional drag” of a probe is evidenced by a distinctive, i.e., unique, electrophoretic mobility of the probe in a non-sieving medium.

A “sieving matrix” or “sieving medium” means an electrophoresis medium that contains crosslinked or non-crosslinked polymers which are effective to retard electrophoretic migration of charged species through the matrix.

The phrase “non-sieving matrix” refers a liquid medium which is substantially free of a mesh, network, or matrix of interconnected polymer molecules.

A “distinctive electrophoretic mobility” of an analyte (e.g., a hydrolytic product comprising a mobility modifier and a reporter but not a partitioner) is evidenced by a distinctive, i.e., unique, electrophoretic mobility of the analyte in a sieving or in a non-sieving matrix.

As used herein, a “distinctive mobility” refers generally to a “distinctive elution characteristic in a chromatographic separation medium” and/or a “distinctive electrophoretic mobility,” as defined above.

The “charge” of a polymer is the total net electrostatic charge of the polymer at a given pH.

An “affinophore” is a soluble ionic carrier comprising one or more affinity ligands. The affinity ligand can be a first member of a binding pair that interacts with the other member of the binding pair, which is referred to herein as a “complementary ligand.”

“Affinophoresis” refers to an electrophoretic separation method employing an affinophore to influence the migration of molecule comprising a complementary ligand, i.e. the binding-pair member that interacts with an affinity ligand of an affinophore. In certain embodiments, the affinophore is immobilized.

The term “reporter” refers to a moiety that, when attached to the compositions of the invention, render such compositions detectable using known detection means, e.g., spectroscopic, photochemical, radioactive, biochemical, immunochemical, enzymatic or chemical means. Exemplary labels include but are not limited to fluorophores, energy-transfer dyes, chromophores, radioisotopes, spin labels, enzyme labels and chemiluminescent labels. Such labels allow direct detection of labeled compounds by a suitable detector, e.g., a fluorescence detector. In addition, such labels include components of multi-component labeling schemes, e.g., a system in which a ligand binds specifically and with high affinity to a detectable anti-ligand, e.g., a labeled antibody or labeled avidin.

“Capillary electrophoresis” means electrophoresis in a capillary tube or in a capillary plate, where the internal diameter of the separation column or thickness of the separation plate is less than 500 microns.

“Separation medium” means a medium typically located within the lumen of a capillary through which an electrophoretic separation is conducted. Exemplary separation media include crosslinked gels, un-crosslinked polymer solutions, or polymer-free solvents, e.g., buffered water. Optionally, separation media may include denaturants such as detergents, e.g., SDS, or organics, e.g., urea or formamide.

“Capillary” or “capillary tube” means tubes or channels or other structure capable of supporting a volume of separation medium. The geometry of a capillary may vary widely and includes tubes with circular, rectangular or square cross-sections, channels, grooved plates, and the like. Capillaries may be fabricated by a wide range of well known technologies, e.g., pulling, etching, photolithography, and the like. An important feature of a capillary for use with the invention is the surface-area-to-volume ratio of the capillary lumen. High values of this ratio permit efficient dissipation of Joule heat produced during electrophoresis. For example, ratios in the range of about 0.4 to 0.04 nm-1 are employed. These ratios correspond to surface-to-volume ratios of tubular capillaries with circular cross-sections having inside diameters in the range of about 10 μm to about 100 μm Capillaries may be formed as individual elements, or as channels formed in a monolithic substrate for example (e.g., Pace, U.S. Pat. No. 4,908,112; Soane and Soane, U.S. Pat. No. 5,126,022). Capillaries include an “inlet end” through which sample analytes are introduced into the lumen of the capillary.

As used herein, the term “spectrally resolvable” in reference to a plurality of reporters means that the reporters are fluorescent dyes having which have fluorescent emission bands that are sufficiently distinct, i.e. sufficiently non-overlapping, that labeled products to which the respective dyes are attached can be distinguished on the basis of the fluorescent signal generated by the respective dyes by standard photodetection systems, e.g. employing a system of band pass filters and photomultiplier tubes, or the like, as exemplified by the systems described in U.S. Pats. No. 4,230,558, and 4,811,218, or the like, or in Wheeless et al., pgs. 21-76, in Flow Cytometry: Instrumentation and Data Analysis (Academic Press, New York, 1985).

3.2 Protease or Protease Family to be Analyzed

In some embodiments, analysis of the specificity of a protease is carried out using a purified enzyme isolated using chromatographic methods and reagents well known in the art. The protease can be isolated from a natural source, e.g. mammalian tissue or cell lines, or from a recombinant source such as, but not limited to, a genetically engineered microorganism overexpressing the protease to be analyzed. In other embodiments, the protease is not isolated but rather is a relatively crude cell extract of a recombinant organism overexpressing the protease is used instead (see e.g. Rosse et al. (2000) J. Comb. Chem. 2: 461).

In some embodiments, the protease activity to be analyzed comprises two or more proteases of the same family, as that term is defined above. In certain instances, the protease activity is isolated from tissue or a cell line in which the plurality of proteases is naturally or recombinantly expressed. In such instances, the protease activity can be isolated from a tissue or from a recombinant organism expressing or overexpressing the plurality of individual proteases. In other instances, each protease can be individually overexpressed in a separate recombinant host and the isolated proteases combined prior to assay.

In some embodiments, the protease activity to be analyzed corresponds to that activity that provides a “fingerprint” of a tissue or sample that is diagnostic of the physiological state of that tissue or sample. In such instances the protease activity of the tissue of interest would encompass that of a plurality of proteases whose hydrolytic activity varies between, e.g., two physiological states of a given tissue such as cancerous as opposed to non-cancerous tissue. Accordingly, in order to develop an assay diagnostic of a disease, a plurality amino acid sequences are identified that are differentially hydrolyzed by a sample derived from diseased tissue as compared with a sample derived from the corresponding non-diseased tissue.

In some embodiments, peptide substrates are identified and then incorporated within a multifunctional tag that is specific or substantially-specific for a target protease or target protease family. In some embodiments, target proteases are identified by analysis of publicly-available genomic sequence information. For example, annotated genomic information is available for, inter alia, the human, mouse, and rat genomes (see e.g. http://www.ncbi.nlm.nih.gov/genome/seq; http://www.genome.ucsc.edu; http://www.sanger.ac.uk/hgp; and http://www.hgsc.bcm.tmc.edu). Accordingly, the coding sequence for such genome-encoded proteases is readily isolated and over-expressed in a recombinant host using methods and reagents well established and widely-known in the art. In one illustrative aspect, a target protease is readily isolated from the recombinant host, e.g. by attaching a hexahistidine tail to the protease and isolating the fusion protein on a chelated-nickel column. In some embodiments, a substantially-unfractionated lysate of the recombinant host overexpressing the target protease can be prepared and used for identification of one or more peptides that are specific or substantially specific for that target protease. A substantially-unfractionated lysate is prepared e.g. by lysing the host cell by freezing and thawing, sonication, blending with glass beads or Dounce homogenization or any other suitable method, and then centrifuging the lysate one or more times to remove unbroken cells and/or cell debris (e.g. see Rosse et al. (2000) J. Comb. Chem. 2: 461, which is hereby incorporated by reference in its entirety).

3.3 Multifunctional Tag Structure

3.3.1 Substrate

The substrate is the portion of the multifunctional tag that comprises a bond cleaved by a target enzyme. Where the target enzyme is a protease, the substrate is a peptide substrate comprising, in certain embodiments, at least two, at least four, at least six, at least eight, at least ten, at least twelve, at least fourteen, at least sixteen, at least eighteen, or at least twenty amino acids joined by peptide bonds. In some embodiments, the amino acids are selected from among the twenty naturally-occurring amino acids incorporated into proteins in vivo. In some embodiments, the peptide substrate may comprise one or more uncommon amino acids including, but not limited to, D-amino acids, norleucine, or one or more amino acid analogues, derivatives, or mimetics such as but not limited to the tyrosine mimetic, (S)-3-(1-hydroxy-p-carboran-12-yl)alanine. In some embodiments, one or more amino acid side chains of the peptide substrate are derivatized with, for example, a reporter, which may be attached directly to the substrate or indirectly via a linker disposed between the reporter and the substrate.

In some embodiments, each peptide substrate incorporated within the multifunctional tags of the present invention is cleaved only by a single protease encoded by the genome of the organism from which the sample to be tested has been obtained, or the amino acid sequence of a peptide substrate can be substantially specific for a target protease. A peptide substrate comprising an amino acid sequence substantially specific for a target protease is one that is hydrolyzed by the target protease with a hydrolytic efficiency that is, in various embodiments, at least about two-fold, three-fold, four-fold, five-fold, or ten-fold greater than that of any other protease present in the sample to be tested. Alternatively, a peptide substrate comprising an amino acid sequence is substantially specific for a target protease if that peptide substrate is hydrolyzed by a sample comprising the target protease with a hydrolytic efficiency that is, in various embodiments, at least about two-fold, three-fold, four-fold, five-fold, or ten-fold greater than the hydrolytic efficiency of the same sample from which the target protease has been removed or from a comparable sample that does not comprise the target protease. Hydrolytic efficiency, as used in this context, refers to the ratio of the maximum rate of hydrolysis of the peptide substrate catalyzed by the protease (or sample comprising a plurality of proteases), designated K_{cat}, to the concentration of a peptide substrate that provides the half-maximal rate of hydrolysis, i.e. the Kₘ; that is, the hydrolytic efficiency, as used herein, refers to the ratio: (K_{cat})/(Kₘ). Therefore, the relative specificity of two proteases or two protease-containing samples for a given peptide substrate can be established by comparing the hydrolytic efficiency of each protease or protease-containing sample for that peptide substrate.

In some embodiments, the peptide substrate comprises an amino acid sequence that is substantially specific with respect to two or more different proteases. In this embodiment, all or substantially all of the members of family, subfamily, or group of proteases exhibit a similar substrate specificity toward particular peptide. Accordingly, in some embodiments, the amount of labeled hydrolytic product detected upon hydrolysis of such a peptide substrate reflects the collective proteolytic activity of that family, subfamily, or group of proteases present in the sample tested.

The terms “peptidase” or “protease,” which terms are used interchangeably herein, describe the set of enzymes that cleave peptide bonds either in a protein or within a fragment thereof, i.e. a peptide. Classification of peptidases or proteases, is difficult in that all such enzymes catalyze the same reaction—hydrolysis of a peptide bond. Differences between and among proteases exist with respect to the position of the cleaved bond within a peptide substrate and amino acid sequences on either side of that bond.

Proteases have been classified into families in view of (1) the different amino acid sequences (generally between two and ten residues) located on either side of the hydrolysis site of the protease, or, alternatively, (2) by comparing the amino acid sequence of the region of each protein believed to be involved in hydrolysis of the peptide bond (see Barrett et al. (2001) J. Structural Biology 134: 95-102; Rawlings et al. (2002) Nucleic Acids Research 30(1): 343-346 and the MEROPS database (http//merops.sanger.ac.uk) described therein which provides available information regarding classification of proteases according to their amino acid sequence and structure as well as according to the amino acid sequences cleaved by those proteases; Barrett et al. (eds.) (1998) HANDBOOK OF PROTEOLYTIC ENZYMES, Academic Press, London; which is incorporated herein by reference). However, for the purposes of the present invention, the former method of classification would be particularly useful in an initial design of peptide substrates, and multifunctional tags comprising those peptide substrates, that would be substantially specific for a family, subfamily, or group of enzymes.

In some embodiments, reaction conditions may be modified either to enhance or to obviate the apparent specificity with which a peptide substrate, and the corresponding multifunctional tag comprising that peptide substrate, is hydrolyzed by a target protease or target protease family. For example, where two proteases hydrolyze the same peptide substrate but with a different hydrolytic efficiency, this substrate specificity can be enhanced by carrying out the hydrolytic reactions using a lower concentration of the peptide substrate, and/or by carrying out the reaction for a shorter period of time. Analogously, substrate specificity can be mitigated or even obviated by carrying out the hydrolytic reactions using a higher concentration of the peptide substrate, and/or by carrying out the reaction for a longer time period. Such mitigation or obviation of peptide substrate specificity is particularly useful where a particular multifunctional tag comprising a peptide substrate is used to measure the total hydrolytic activity of a family or group of proteases in a sample where individual members of that family or group all hydrolyze the substrate but with differing hydrolytic efficiency.

Although in many instances, the amino acid sequence of a peptide cleaved in vivo by a protease has been identified (Barrett et al. (2001) J. Structural Biology 134: 95-102; Rawlings et al. (2002) Nucleic Acids Research 30(1): 343-346; MEROPS database (http//merops.sanger.ac.uk); and Barrett et al. (eds.) (1998) HANDBOOK OF PROTEOLYTIC ENZYMES, Academic Press, London), that amino acid sequence may not be preferred or even useful in the present invention. For example, using methods to be discussed below, an amino acid sequence was identified that was hydrolyzed over five-thousand-fold more efficiently in vitro by tissue plasminogen activator than a peptide comprising the amino acid sequence of the natural substrate, plasminogen, hydrolyzed by that enzyme in vivo (Ding et al. (1995) Proc. Natl. Acad. Sci. USA 92: 7627-31).

The efficiency with which a specific peptide bond is hydrolyzed by a specific protease may be influenced by the amino acid sequence within which that peptide bond is found or to which it is appended. The number of potential, random amino acid sequences that can be generated is very large; i.e. 160,000 different tetrapeptides and more than twenty-five billion octapeptides can be designed using a set of twenty amino acids. However a number of methods, including four described below, are available that are useful for the construction of large collections of peptides, with each peptide having a defined amino acid sequence. Such peptide collections are readily analyzed using methods disclosed herein to identify those amino acid sequences that are either specific or at least substantially specific for a target protease or for a target protease family. Moreover, once one or more large collections of peptides are assembled, using e.g. one or more of the methods described in Sections 5.3.1.1-5.3.1.4 below, those collections can be repeated “mined” for peptides that are specifically or substantially-specifically hydrolyzed by each different target protease or target protease identified during, e.g. detailed analysis of the human genome.

Amino acids that are involved in the recognition and binding of a peptide substrate by a protease may lie either “upstream” (toward the amino terminus of the peptide substrate) or “downstream” (toward the carboxyl terminus of the peptide substrate) of the peptide bond that is hydrolyzed. By convention, the four, e.g., amino acids downstream of the peptide bond hydrolyzed by a protease are referred to as “prime-side” amino acids and are designated P₁′-P₄′, where the numbering begins with the amino acid involved in the peptide bond hydrolyzed. Similarly, the four, e.g., amino acids upstream of the peptide bond hydrolyzed by a protease are referred to as “non-prime-side” amino acids and are designated P₁-P₄, where the numbering again begins with the amino acid involved in the peptide bond hydrolyzed. Therefore, an eight amino-acid long peptide hydrolyzed by a protease could have the following “structure:” NH₂-P₄-P₃-P₂-P₁-P₁′-P₂′-P₃′-P₄′-COOH, where the peptide bond hydrolyzed by the protease is that joining amino acids P₁ and P₁′.

In some embodiments, the peptide bond hydrolyzed by a target protease or target protease family is formed using the carboxylic acid moiety of the carboxy-terminal amino acid of the peptide substrate. That is, such peptide substrates do not include any “downstream” amino acids.

In some embodiments, the peptide bond hydrolyzed by a target protease or target protease family is formed using the amine moiety of the amino-terminal amino acid of the peptide substrate. That is, such peptide substrates do not include any “upstream” amino acids.

In some embodiments, the peptide bond hydrolyzed by a target protease or target protease family is embedded within the peptide substrate. That is, such peptide substrates include at least one amino acid upstream and at least one amino acid downstream of the peptide bond hydrolyzed by the target protease or target protease family.

3.3.1.1 Non-Prime Side Analysis

Non-prime side analysis refers to determination of the amino acid sequence preference for a protease upstream of, i.e. amino-terminal to, a peptide bond hydrolyzed by that protease. One method useful in such a determination is referred to as positional scanning. In this approach, e.g., a collection of tetrapeptides are assembled which have the general structure: (Acetyl)-NH₂-P₄-P₃-P₂-P₁-C(O)—(NH-Leaving Group). Hydrolysis of the caboxy-terminal peptide bond by a protease releases the leaving group which is highly fluorescent as compared with the non-hydrolyzed peptide substrate. Leaving groups useful in such methods include 7-amino-4-methylcoumarin (AMC) and 7-amino-4-carbamolylmethylcoumarin (ACC). The latter compound is particularly useful in that it (1) has a quantum yield approximately three fold greater than that for AMC, and (2) is readily attached directly to a solid support, thereby facilitating the synthesis of peptide-ACC substrates (see e.g. Maly et al. (2002) J. Org. Chem. 67: 910-15, and Harris et al. (2000) Proc. Natl. Acad. Sci. USA 97(14): 7754-59, both of which are hereby incorporated by reference in their entirety). Four pools of peptides are synthesized within which one amino acid position of the peptide (i.e. either P₄, P₃, P₂, or P₁) is “scanned.” Therefore, each pool, in turn, comprises e.g. 20 subsets of peptides. Within each subset, one amino acid position (e.g. P₁) is specifically defined while each of the remaining positions (P₄, P₃, and P₂) is synthesized as a mixture of, generally, 19 proteinogenic amino acids (cysteine is usually excluded). Accordingly, in this illustration, the 20 subsets differ from one another only with respect to the amino acid present in position P₁. Each subset therefore includes a total of (19)³ or 6589 different peptides, while the 20 subsets within each pool include a total of (19)⁴ or 131,780 different tetrapeptide sequences. Since four positions (P₄, P₃, P₂, and P₁) of the peptide sequence are to be scanned in this illustration, a total of 527,120 tetrapeptides are employed. In one approach using this method, each of the 20 subsets of each of the four pools is synthesized and analyzed separately. An aliquot of each subset is hydrolyzed by a protease and the rate of release of the fluorescent leaving group determined for each of subset. Since the peptides within each subset differ only with respect to the amino acid present at e.g. position P₁, the relative rates of hydrolysis observed are a reflection of the preference for the protease in question for that amino acid at that position. The combined data obtained upon analysis of the rate of hydrolysis of each of the 80 subsets indicates which amino acid is preferred by the protease at each of positions P₄, P₃, P₂, and P₁.

Methods and reagents for the synthesis of peptide substrates comprising suitable leaving groups that are useful in positional scanning methods as well as methods and equipment useful for analyzing the hydrolysis of such substrates by proteases are well known in the art (see e.g., Richardson, P. L. (2002) Current Pharmaceutical Design 8: 2559-81; Maly et al. (2002) J. Org. Chem. 67: 910-15, and Harris et al. (2000) Proc. Natl. Acad. Sci. USA 97(14): 7754-59; Rano et al. (1997) Chemistry & Biology 4: 149-155; Thornberry et al. (1997) J. Biol. Chem. 272(29): 17907-11; and Mathieu et al. (2002) Molec. Biochem. Parasitol. 121: 99-105, each of which is hereby incorporated by reference in its entirety).

In certain embodiments, a peptide substrate is constructed and incorporated within the multifunctional tags of the present invention, which includes at each position the amino acid most preferred by the protease as identified by positional scanning. In other embodiments, however, the peptide substrate may include one or more “less preferred” amino acids at one or more specific positions within the peptide substrate, where such a “sub-optimal” peptide substrate would provide enhanced specificity and selectivity with respect to one or more other proteases found in the particular sample to be analyzed.

3.3.1.2 Prime Side Analysis

In those instances in which a protease cannot hydrolyze the peptide bond formed between the carboxyl-terminal carboxylate moiety of a peptide and the detectable leaving group, the non-prime side analysis of Section 5.2.1.2 cannot be used to identify an amino acid sequence useful for designing a peptide substrate specific or substantially specific for a protease or protease family. In such instances, a preferred amino acid sequence contiguous with a peptide bond hydrolyzed by a protease or protease family can be identified using a method referred to as “prime-side” analysis. This method is analogous to the non-prime side method of the preceding section with respect to the use of positional scanning. However, the peptide substrates employed in prime-side analysis have the following general structure: Leaving Group —C(O)—NH-P₁′-P₂′-P₃′-P₄′-C(O)OH. One leaving group useful in this method is 5-fluorosalicyclic acid. Hydrolysis of the peptide bond joining 5-fluorosalicyclic acid to the peptide releases 5-fluorosalicyclic acid, which then interacts with EDTA-complexed terbium ion to provide a fluorescent complex. Accordingly, determination of preferred amino acid sequences downstream (toward the carboxyl terminus of the peptide substrates) of the peptide bond hydrolyzed by a protease can carried out using the general positional scanning described in the preceding section except that the leaving group is attached via a peptide bond, to the amino-terminus of the peptides to be used as substrates. Again, reagents and methods useful for carrying out prime side analysis to identify those amino acids preferred in positions, e.g. P₁′-P₂′-P₃′-P₄′ for hydrolysis by a protease or protease family of interest are well known in the art (see e.g. Barrios et al. (2002) Bioorg. Med. Chem. 12: 3619-23, which is hereby incorporated by reference in its entirety).

3.3.1.3 Fluorescent Resonance Energy Transfer (FRET) Analysis

In many instances, the peptide binding region of an endopeptidase can be sufficiently extended, such that amino acids found both upstream and downstream of the peptide bond hydrolyzed can influence the specificity and selectivity of a protease for a peptide substrate. Accordingly, in certain, instances, it would be preferred that identification of the amino acid sequence specific for or substantially specific for a protease or protease family of interest be carried out using substrates comprising both non-prime side and prime-side amino acids.

One approach to the simultaneous identification of preferred amino acid sequences both upstream and downstream of a peptide bond hydrolyzed by a protease or protease family is based on fluorescent resonance energy transfer. In this approach, combinatorial synthesis methods are used to construct a collection of all possible peptides of up to about six amino acids in length that are attached to a solid surface or insoluble polymer. In this embodiment, each peptide comprises both a fluorescent moiety (donor) as well as another moiety (acceptor) that quenches the fluorescence of the donor. In one approach, the peptides are assembled on beads, which are formed from polyethyleneglycol-poly-(N,N-dimethylacrylamide)copolymers that allow access of proteases into the interior of beads (see e.g. U.S. Pat. No. 5,352,756 to Meldal, which is hereby incorporated by reference in its entirety). The donor moiety (e.g. ortho-aminobenzamide) can be covalently bound, for example to the side-chain amino group of a lysine residue, which serves as the carboxy-terminal amino acid of the peptide chains, and which is attached to the resin, to provide a labeled resin. The labeled resin is divided into 20 portions, each of which is reacted with one of the Fmoc derivatives of the 20 proteinogenic amino acids. After the coupling reactions were complete, the 20 portions of resin are thoroughly mixed and divided again into 20 equal portions for addition of the second amino acid residue to the growing peptide chains. Each coupling, deprotection, and mixing cycle is repeated until the desired peptide is constructed. Finally, an amino-terminal residue comprising an acceptor or quencher (e.g. 3-nitrotyrosine) is covalently attached to all of the peptide chains bound to the resin beads.

Each resin bead carries multiple copies of a single peptide chain, while the collective population of beads derivatized in this manner comprises more than 10⁷ different peptides. Moreover, these resin beads comprising both the donor and quencher pair are essentially non-fluorescent. Hydrolysis of a resin-bound peptide chain by a protease or protease family eliminates the quenching effect of the amino-terminal 3-nitrotyrosine and the resulting bead is highly fluorescent, readily detected and can physically separated from the remainder of the non-fluorescent resin beads carrying non-hydrolyzed peptide chains either by hand or using an automated separator.

In view of number of peptide chains bound to each resin bead and the observation that only a portion of those chains are hydrolyzed on the fluorescent beads selected from the population, it has been demonstrated in the art that amino acid sequence analysis performed on a selected fluorescent bead will provide not only the sequence of the resin-bound peptide prior to hydrolysis but also the identity of the peptide bond hydrolyzed by the protease or protease family. Peptides identified in this manner can be re-synthesized and subjected to solution-phase hydrolysis by the protease, protease family, or other sample comprising one or more catalytically-active proteases, in order to determine values for Kₘ, Vₘₐₓ, and K_{cat} for each peptide substrate/protease combination using standard Michaelis-Menten kinetic analyses that are well known in the art. Moreover, a hierarchy of protease-specific or substantially-protease-specific amino acid sequences can be established using this method that would include both optimal peptide substrates as well as, in certain embodiments, sub-optimal peptide substrates, (as defined by (K_{cat}/Kₘ)). Such sub-optimal peptide substrates could be incorporated within the multifunctional tags of the present invention to provide substrates having greater specificity and selectivity with respect to a specific protease or protease family. Methods and reagents useful for the combinatorial assembly of such resin-bound fluorescence-quenched peptide libraries as well as the use thereof for identification of protease-specific amino acid sequences are known in the art and include, but are not limited to those described by Meldal et al. (1994) Proc. Natl. Acad. Sci. USA 91: 3314-18; Meldal, M. (2002) Biopolymers (Peptide Science) 66: 93-100; Meldal, M. (1998) Methods Mol. Biol. 87: 51-82.; and Meldal et al. (1998) J. Peptide Sci. 4: 83-91, each of which is hereby incorporated by reference in its entirety.

3.3.1.4 Phage Display

Another method for screening peptide substrates for the identification of amino sequences specific or substantially specific for a protease or protease family involves the use of phage display methodology. Phage display procedures involve the construction of very large libraries of recombinant phage displaying random peptide substrates on the surface of the phage particle. Peptide substrate sequences are inserted between the amino terminus of a phage capsid protein and a protein sequence, referred to as a “tether,” which is a member of a binding pair. Hydrolysis of the peptide substrate separates the infectious phage particle from the tether allowing rapid and specific separation of phage particles comprising an “efficient” peptide substrate from those phage particles that do not using methods described below.

More specifically, amino acid sequences specific or substantially specific for a protease or protease family can be identified by screening libraries of recombinant M13 or fd phage of E. coli expressing a chimeric version of the gene III “pilot” protein of either phage. Five copies of the pilot protein are found at one end of these filamentous phage particles. Using standard recombinant DNA methodology well known in the art, the gene III coding sequence is modified to include a tether coding region that will be fused to the amino terminus of the pilot protein.

The tether, in certain embodiments, is a member of a specific, stable binding pair, which bind to the second member of the binding pair that has been immobilized on a solid surface. The tether can be, for example, (1) a hexahistidine sequence that is tightly bound onto columns to which chelated nickel is bound, (2) a peptide comprising one or a plurality of peptide antigens that are bound by one or more monoclonal antibodies immobilized on a column, matrix, or surface carrying bound protein A, or (3) a ligand bound by its cognate receptor such as a tight-binding human growth hormone (hgh) tether that can be bound by immobilized hgh-receptor.

The population of peptide sequences is encoded, for example, by a population of DNA fragments that is inserted into the chimeric gene III coding region, between the coding region for the pilot protein and the coding region for the tether. One, non-limiting approach to the construction of a population of DNA fragments encoding for example, all possible hexametric amino acid sequences involves the synthesis of three oligonucleotides. The first comprises three regions: a 5′-terminal region, a 3′-terminal region, and a central region. the 5′-terminal and 3′-terminal regions consist of defined nucleotide sequences which could be used to encode for example, spacer or linking peptides such as Gly-Pro-Gly-Gly and Gly-Gly-Pro-Gly respectively, which could disrupt protein structures extending from either the pilot or the tether protein domains, and which could also provide flexibility to the target peptide. The central region is synthesized for example as an 18 nucleotide sequence represented as six consecutive (NNK) codons, where N represents and equimolar mixture of G, A, T, and C, while K represents an equimolar mixture of G and T. The second oligonucleotide comprises a DNA sequence complementary to the 5′-terminal region of the first oligonucleotide while the third oligonucleotide comprises a DNA sequence complementary to the 3′-terminal region of the first oligonucleotide. Accordingly, annealing the three oliognucleotides provides a gapped duplex in which the 5′-terminal and 3′-terminal regions exist as duplex structures bracketing the central, single-stranded (NNK)₆ region. The ends of each of the three oligonucleotides are designed and constructed to include appropriate 5′-protruding, 3′-protruding, or flush-ended structures to facilitate directed, in-frame insertion of the gapped duplex into the chimeric pilot protein-tether coding sequence.

The recombinant gene assembled in this manner, encodes a fusion protein comprising the pilot protein, a six amino acid peptide substrate, the tether, and, if desired, linking or spacer peptide sequences disposed between the pilot protein and the peptide substrate, and between the peptide substrate and the tether. However, as would be apparent to those of ordinary skill in the art, there are many different approaches that could be used to assemble recombinant genes encoding such chimeric proteins (see e.g. Smith et al. (1995) J. Biol. Chem. 270(12): 6440-6449; Matthews et al. (1993) Science 260: 1113-1117; and Cwirla et al. (1990) Proc. Natl. Acad. Sci. USA 87: 6378-6382, each of which is hereby incorporated by reference in its entirety).

In certain embodiments, phage display methods can be used for identification of amino acid sequences specific or substantially specific for a protease or protease family using, for example, what are referred to as “monovalent” or “polyvalent” systems. Monovalent phage display systems, as described by Matthews et al. (Matthews et al. (1993) Science 260: 1113-1117) employ recombinant phagemid vectors that include a recombinant gene encoding a tripartite chimeric protein comprising a pilot protein, peptide substrate, and tether. Phage particles are generated by infecting an E. coli host strain carrying the phagemid with helper phage. It has been estimated that only approximately 10% of the population of phage particles generated according to this method display the chimeric surface protein, and even in those phage particles that do, there is only a single copy of that chimeric surface protein. Consequently, the desired sub-population of phage particles displaying the chimeric surface protein is first adsorbed, via binding of the tether to its immobilized binding partner, to a surface. After one or more wash steps to remove wild-type, non-recombinant phage, the immobilized recombinant phage are exposed to the protease to be analyzed. Hydrolysis of the peptide substrate by the protease releases the infectious phage particles which are collected and amplified. This series of steps is repeated allowing the isolation of recombinant phage particles encoding peptide substrates efficiently hydrolyzed by the protease analyzed.

In other embodiments, polyvalent systems are generated by genetically engineering the double-stranded replicative form of a filamentous virus such as M13 or fd to provide infectious, recombinant phage, with each phage particle displaying five copies of each chimeric pilot protein comprising, at the amino-terminus of the chimeric protein, a peptide substrate, generally involving five or six amino acid residues, and a tether (see e.g. Smith et al. (1995) J. Biol. Chem. 270(12): 6440-6449; Ke et al. (1997) J. Biol. Chem. 272(33): 20456-20462; Ding et al. (1995) Proc. Natl. Acad. Sci. USA 92: 7627-31; Ke et al. (1997) J. Biol. Chem. 272(26): 16603-16609; and Cwirla et al. (1990) Proc. Natl. Acad. Sci. USA 87: 6378-6382). Phage display libraries constructed in this manner encompass phage that display, collectively, approximately 10⁸ different peptide substrates. The phage library is contacted with the protease for which a specific peptide substrate is sought and recombinant phage carrying non-hydrolyzed peptide substrates and the attached tether are separated by binding to the surface-immobilized binding partner. Non-bound infectious recombinant phage particles are amplified to provide a first population of recombinant phage expressing peptide substrates preferentially cleaved by the subject protease. This first population of recombinant phage can then be subjected to another enrichment cycle in which the first population of phage is subjected to a second round of hydrolysis with the subject protease, removal of phage particles comprising non-hydrolyzed peptide substrates and a tether, and amplification to provide a second population of recombinant phage expressing peptide substrates preferentially cleaved by the subject protease.

The selectivity of the amino acid sequences hydrolyzed by a specific protease identified using such phage display methods can be enhanced by decreasing the amount of the subject protease used to hydrolyze the displayed peptide substrates as well as by decreasing the duration of the hydrolysis reactions in one or more enrichment cycles.

Where two or more proteases are known to exhibit overlapping preferences for peptide substrates, phage display methods are modified in order to identify amino acid sequences specific or substantially specific for each of the proteases in question. In this embodiment, a polyvalent phage display library is subjected to, e.g., one to three rounds of (a) hydrolysis, (b) removal of phage expressing non-hydrolyzed peptide substrates, and (c) amplification of those phage expressing peptide substrates hydrolyzed by the first protease. The population so generated is, therefore, substantially enriched in amino acid sequences preferentially hydrolyzed by the first protease.

This enriched population is then hydrolyzed with the second protease that has a substrate specificity that overlaps that of the first protease. In this instance, those phage expressing non-hydrolyzed peptide substrates and a tether are retained, e.g. by immobilization mediated by the interaction between the tether and its corresponding binding partner attached to a solid surface. The immobilized population of phage, therefore has been depleted of those sequences efficiently hydrolyzed by the second protease. Subsequent hydrolysis of this “depleted” population with the first protease releases phage particles encoding peptide sequences that are efficiently hydrolyzed by the first protease but are not efficiently hydrolyzed by the second protease. Repetition of these steps, including that for depletion of sequences hydrolyzed by the second protease, can provide an amino acid sequence specific or substantially specific for the first protease but not the second.

In a similar manner, an amino acid sequence specific or substantially specific for the second protease but not the first protease is identified by generating phage populations enriched in peptide sequences preferentially hydrolyzed by the second protease but depleted of those hydrolyzed by the first protease. Reagents and methods useful in this embodiment are described by Ke et al. and Ding et al. (see e.g. Ke et al. (1997) J. Biol. Chem. 272(33): 20456-20462; Ding et al. (1995) Proc. Natl. Acad. Sci. USA 92: 7627-31; and Ke et al. (1997) J. Biol. Chem. 272(26): 16603-16609, each of which is hereby incorporated by reference in its entirety).

In another aspect of this embodiment, the first and the second proteases for which specific amino acid sequences are to be identified are not pure proteins but correspond to extracts comprising a plurality of proteases that are present in a sample isolated from a first, normal tissue, and a second, diseased tissue. In this embodiment, one or more amino acid sequences are identified as specific or substantially specific for one or more proteases that are catalytically active in normal tissue but not in the corresponding diseased tissue. Similarly, one or more amino acid sequences are also identified that are specific or substantially specific for one or more proteases that are catalytically active in diseased tissue but not in the corresponding normal tissue. In certain embodiments, the first and second tissue samples are separately contacted with a first or a second composition comprising a set of multifunctional tags comprising peptide substrates efficiently cleaved by proteases found in the first or in the second tissue sample. In one aspect of this embodiment, the multifunctional tags of the first and second compositions differ only with respect to the reporter used; e.g., the first and second reporters can be spectrally-resolvable fluorescent dyes. Therefore, in this aspect, the products of both reactions can be combined, fractionated, and analyzed together.

3.3.1.5 Selection and Testing of Peptide Substrates

The methods of Sections 3.3.1.1 to 3.3.1.4, above, are used to identify a set of peptides that are readily cleaved by a target protease or target protease family. Each member of this set is generally further analyzed to determine relevant kinetic parameters, e.g. the Km, Vmax, Kcat and hydrolytic efficiency (Km/Kcat), for hydrolysis of that peptide by a specific target protease or protease family. These parameters are readily determined by those of skill in the art generally according to the principles of Michaelis-Menton enzyme kinetics.

In specific embodiments, derivatives of each member of the set of peptides readily cleaved by a target protease or protease family are prepared generally according to the methods described above in Sections 3.3.1.1 to 3.3.1.3, to provide fluorescent products. Hydrolysis of each labeled peptide is carried out in an aqueous, buffered medium, generally at a temperature within the range of from about 20° C. to about 40° C. Individual hydrolytic reactions are carried out at different concentrations of the labeled substrate, often using 96-well or 384-well microtiter plates and analyzed using a commercially-available automated microplate reader, such as the SpectraMax 250, Specramax 384, or VERSAmax Tunable Microplate Reader of Molecular Devices (Molecular Devices Corporation, Sunnyvale, Calif.). Data obtained are analyzed manually, using e.g. Lineweaver-Burk plots well known in the art or, more conveniently, using software designed for such applications, including e.g. Softmax (Molecular Devices Corporation, Sunnyvale, Calif.), SigmaPlot, including the Enzyme Kinetics Module option (SPSS Science Inc., Chicago, Ill.), Enzyme Kinetics (Chemistry-Software.com of Emedia Science Ltd., Birkenhead, Wirral, United Kingdom), and EnzFitter (Biosoft, Ferguson, Mo.).

The data collected in this manner, particularly the hydrolytic efficiency of each candidate peptide for each target protease, facilitate the selection of a peptide substrate that is specific or at least substantially specific and/or selective for each protease or protease family represented within a sample to be analyzed.

Peptides identified in this manner as specific or substantially specific for a target protease or protease family are then assembled within the multifunctional tags of the invention. In certain embodiments, the peptide substrate may include, where necessary or desired, one or more additional amino acids to increase the flexibility of and/or to obviate conformational constraints on the structure of the peptide.

3.3.2 Mobility Modifier

The multifunctional tags of the present invention comprise a peptide substrate, and, attached thereto either directly or indirectly via a linker, a mobility modifier, at least one reporter and a partitioner. Hydrolysis of a multifunctional tag of the present invention by a specific or substantially-specific protease or protease family provides a hydrolytic product comprising the mobility modifier and reporter but not the partitioner. In particular embodiments of the present invention, a plurality of proteases or protease families present in a sample are detected simultaneously by contacting that sample with a plurality of different multifunctional tags each of which is specific or substantially-specific for a particular target protease or target protease family to be detected. In these embodiments a plurality of different products are obtained where each is diagnostic for a target protease or protease family. Separation, detection, and identification of each labeled hydrolytic product (i.e. comprising a mobility modifier and reporter but not a partitioner) is accomplished using a mobility-dependent analysis technique. Resolution of each particular, different labeled hydrolytic product from other labeled hydrolytic products is mediated by the mobility modifier component thereof, which confers a distinctive mobility, e.g. a distinctive electrophoretic mobility, upon that particular labeled hydrolytic product when separated using a mobility-dependent analysis technique.

In certain embodiments, mobility-modifying polymer chains are attached to the peptide substrates, either directly or through an intervening linking group. In some embodiments, the mobility-modifier comprises a polymer such as, but not limited to polyethylene oxide, polyglycolic acid, polylactic acid, oligosaccharide, polyurethane, polyamide, polyamine, polyimine, polysulfonamide, polysulfoxide, or block copolymer thereof, including polymers composed of units of multiple subunits linked by a charged or uncharged linking group. In certain embodiments, the mobility-modifier is a nucleic acid, e.g., an oligodeoxyribonucleotide or a peptide nucleic acid. Therefore, such compositions also embody polymer chains in the form of copolymers or block polymers, of, for example, polyethylene oxide and a polyamine and having one or more charged or uncharged linkers joining adjacent monomer units.

In some embodiments, mobility-modifying polymers are or comprise polyoxides or polyethers. In this context, the term polyoxide is used to denote polymers with oxygen atoms in the main chain, particularly those with monomer units of the type —[O—(CH₂)ₙ]— where n is an integer selected from the range of 1 to 15. In certain embodiments n is selected from the range of 2 to 6, and in other embodiments, n=2, together with their derivatives. Linear polyoxides applicable to the composition include, for example, poly(methylene oxide), poly(ethylene oxide), poly(trimethylene oxide), poly(tetramethylene oxide), poly(pentamethylene oxide), poly(hexamethylene oxide), and poly(heptamethylene oxide). Branched polyoxides provide additional moieties available for mobility-modification by, in some cases, imparting to the mobility-modified peptide substrate or hydrolytic product thereof a translational frictional drag that is different than that provided by a linear polymer chain. Branched polymers, for example poly(propylene oxide) which are appreciably soluble in aqueous solvents, are used in certain embodiments. Other applicable branched polymers include poly(acetaldehyde), and poly(but-1-ene oxide).

In some embodiments, the mobility-modifying polymer is a monodisperse linear polyoxide of polyethyleneoxide (PEO) because of its high degree of solubility in a variety of aqueous and organic solvents. Moreover, the chemistry of polyethylene oxides and methods of use thereof for mobility-modifying chemical and biological compounds are well known in the art (see e.g. Grossman, P. D. et al., U.S. Pat. No. 5,777,096; U.S. Pat. No. 5,470,705; U.S. Patent Application Publication No. 2002/0182602 A1; WO 00/55368; WO 01/49790; and WO 02/83954, each of which is hereby incorporated by reference in its entirety). Accordingly, those skilled in the art can readily vary the number of polyethylene units in the mobility-modifying polymer, as well as the nature and charge of the linking groups used to join adjacent monomer units, to impart a distinctive electrophoretic mobility to the mobility-modified hydrolytic product of each protease-specific peptide substrate. This difference in electrophoretic mobility can, but need not be, the result of a distinctive ratio of charge to translational frictional drag.

In addition, the mobility-modifying polymers of the embodiment may further comprise functional groups, such as a hydroxyl, sulfhydral, amino or amide group. These functional groups permit attachment of various reporter molecules, ligands, or other polymer chains, including additional mobility-modifying polymer chains. Protecting groups may be present on such functional groups when the mobility-modifying polymer is being coupled to the peptide substrate, or during reaction of other functional groups with the peptide substrate. Chemical moieties suitable for protecting specific functional groups, including methods for their removal, are well known in the art which also provides ample guidance for selecting the appropriate protecting reagents (see e.g. Greene and Wuts, (1991) Protective Groups in Organic Synthesis, 2^{nd} ed., John Wiley & Sons, Inc., New York). For example, hydroxyl groups are protectable with acid labile groups such as dimethoxytrityl (DMT), or with base labile group such as fluorenyl methyl chloroformate (Fmoc).

Another aspect of the present teachings involves linking groups that join monomer units of the mobility modifier to each other. In some embodiments, that linking group is a phosphate triester, phosphonate, phosphoamidate, phosphothioester or phosphodithioate linkage. Phosphonate and phosphate triester linkages permit attachment of other chemical constituents to the phosphorous atom to effect further differences in the ratio of charge to translational frictional drag between mobility-modified hydrolytic products of each different peptide substrate of a plurality of multifunctional tags. Thus, one embodiment includes alkylphosphonate linkages, such as methyl phosphonate.

In some embodiments, the linkage is a neutral phosphate triester, wherein the free ester has attached various chemical groups so as to render the linker uncharged, such as alkyls, functionalized alkyls, or polymers. When the chemical group is an alkyl, the compound may be a linear or branched alkyl, generally a lower alkyl group. Linear alkyls include, but are not limited to, methyl, ethyl, propyl, or butyl groups, while branched alkyls include, but are not limited to, isopropyl or tertbutyl groups. However the chemical groups attached to the free ester are generally limited to those groups that are stable to all steps of conventional phosphoramidite chemistry, including deprotection steps and especially to the procedures and conditions required for the deprotection of protected amines, such that the resulting linkage is an uncharged phosphate triester. Therefore, when such groups are alkyl, the group is generally an alkyl other than methyl, for example, C₂-C₆ linear alkyl, since mono-methyl phosphate triesters tend to be less stable than higher-order alkyl phosphate triesters. The alkyl group may also have attached functional moieties, such as reporters, ligands or biotin molecules. Such reporter molecules include but are not limited to fluorescent, chemiluminescent or bioluminescent molecules, while ligands include, but are not limited to, molecules such as cholesteryl, digoxigenin, 2,4 dinitrophenol, phenyl boronic acid moieties, and biotin. When the chemical group is a polymer, the same types of polymers set forth above, including but not limited to polyoxides, polyamides, polyamines, polyamides, polyimines, polysaccharides, and polyurethanes, function as suitable substituents.

The mobility-modifier may be covalently attached to the peptide substrate of the multifunctional tag at the amino-terminal end of the peptide substrate, the carboxyl-terminal end of the peptide substrate, or on a side chain of one of the amino acids of the peptide substrate. Exemplary polymer chains that are attached to the peptide substrate include those formed of polyethylene oxide, polyglycolic acid, polylactic acid, oligosaccharide, polyurethane, polyamids, polysulfonamide, polysulfoxide, and block copolymers thereof, including polymers composed of units of multiple subunits linked by charged or uncharged linking groups. In some embodiments, each mobility-modifying-polymer chain (or elements forming a mobility-modifying polymer chain) imparts to the hydrolytic product of each peptide substrate to which it is attached, a distinctive mobility under chromatographic or electrophoretic conditions or other, suitable, mobility-dependent analysis technique. IN some embodiments, the distinctive mobility is a distinctive electrophoretic mobility resulting from a distinctive ratio of charge/translational frictional drag that can be achieved by differences in the lengths (number of subunits) of the polymer chain as well as by the inclusion of one or more charged and/or uncharged moieties, particularly as linking groups used to join adjacent monomer units of the mobility-modifying polymer.

Generally, the mobility-modifying polymers may be homopolymers, random copolymers, or block copolymers, preferably in a linear configuration. Alternatively, the mobility-modifying polymer chains may be in comb, branched, or dendritic configurations. In addition, although the invention is described herein with respect to a single polymer chain attached to an associated peptide substrate at a single point, the invention also contemplates peptide substrates that are derivatized by more than one polymer chain element, where the elements collectively form the mobility-modifier.

In some embodiments, polymers are those which ensure that the multifunctional tag and the hydrolytic product of the peptide substrate are soluble in an aqueous medium. The mobility-modifying polymers should also not adversely affect hydrolysis of the peptide substrate by a target protease. Particularly, where the peptide substrates are highly charged, the mobility-modifying polymer chains are generally uncharged.

In another embodiment the polymers can be dendritic polymers, such as polymers containing polyamidoamine branched polymers (Polysciences, Inc., Warrington, Pa.), for example.

Methods for synthesizing selected-length polymer chains and covalently attaching those chains to a peptide substrate are described below in Sections 5.4.2 and 5.4.4, as well as in U.S. Pat. No. 5,777,096; U.S. Pat. No. 5,470,705; U.S. Patent Application Publication No. 2002/0182602 A1, WO 00/55368, WO 01/49790, and WO 02/83954, each of which is hereby incorporated by reference in its entirety.

In some embodiments, the mobility modifier comprises one or more ligands that interact with a binding partner attached to an immobilized polymer, i.e. immobilized affinophore, and thereby confer a distinctive mobility, e.g. a distinctive electrophoretic mobility, upon the labeled hydrolytic product of which the mobility modifier is a component.

The nature of the mobility modifier used for the construction of each multifunctional tag is dependent upon the nature of the partitioner and the overall nature of that multifunctional tag. For example, where the mobility-dependent analysis technique to be used is electrophoresis, e.g. capillary electrophoresis, the multifunctional tag is designed so as to comprise a mobility modifier carrying a net positive charge and a partitioner carrying a larger net negative charge such that that the overall net charge on the multifunctional tag is negative. Accordingly, proteolytic hydrolysis of such a multifunctional tag provides a positively-charged, labeled hydrolytic product that will migrate toward the cathode during electrophoretic analysis while the negatively-charged multifunctional tag, as well as any hydrolytic product comprising the partitioner which would also be negatively-charged, would migrate in the opposite direction, i.e. toward the anode. In other embodiments, the mobility modifier carries a net negative charge, while the partitioner and multifunctional tag carry a net positive charge.

In some embodiments, the mobility modifier comprises a ligand, which can be used for affinity-based separations, such as but not limited to affinophoresis. In one aspect of this embodiment, the mobility modifier comprises an affinity ligand that will interact with a second, complementary affinity ligand present during electrophoretic separation. For example, separation of hydrolytic products containing a reporter and mobility modifier comprising a first affinity ligand, but not the partitioner, can be fractionated using capillary affinity electrophoresis in which a second, complementary affinity ligand is attached, e.g., to the inner wall of the capillary. In further aspects of this embodiment, the second affinity ligand can be attached to a soluble, highly-charged polymer (e.g. diethylaminoethyl dextran, (DEAE-dextran), polyacryloyl-β-alalnyl-β-alanine, or succinyl-poly-L-lysine), present in the electrophoresis buffer. In still further aspects of this embodiment, the second, complementary affinity ligand is immobilized within the capillary tube in the form of, as non-limiting examples, a cross-linked protein matrix or a hydrogel containing the second affinity ligand.

In some embodiments, the interaction between the first and second affinity ligand is characterized by a dissociation constant of about 10⁻² to about 10⁻⁷ M⁻¹, from about 10⁻³ to about 10⁻⁶ M⁻¹, or from about 10⁻⁴ to about 10⁻⁵ M⁻¹. Representative, non-limiting, examples of such affinity ligand pairs include (1) polyanion-polycation, (2) antibody-antigen, (3) lectin-saccharide, (4) phenyl boronic acid derivate-diol-containing molecule or salicylate derivative, and (5) nucleic acid-complementary nucleic acid where either or both can be a peptide nucleic acid, (see e.g., Buijt-van Duijn et al. (2000) Electrophoresis 21: 3905-18; Shimra et al. (1996) Meth. Enzymol. 271: 203-218; Gram et al. (1992) Proc. Natl. Acad. Sci. USA 89: 3576-80; Barbas et al. (1991) Proc. Natl. Acad. Sci. USA 88: 7978-82; Strachan et al. (2002) FEMS Microb. Lett. 210: 257-61; Kang et al. (1991) Proc. Natl. Acad. Sci. USA 88: 4363-66; Ljungberg et al. (1998) Electrophoresis 19: 461-64; Shimura et al. (1998) Electrophoresis 19: 397-402; Wiley et al. (2001) Bioconjugate Chem. 12: 240-50; Stolowitz et al. (2001) Bioconjugate Chem. 12: 229-239; Le et al. (1997) J. Chromatography A 781: 515-22; Kim et al. (2001) J. Chromatography 754: 97-106; Kim et al. (2001) J. Chromatography B 754: 97-106; Hong et al. (2001) J. Chromatography B 752: 207-16; Wu et al. (1998) Electrophoresis 19: 2650-53; VanderNoot et al. (1998) Electrophoresis 19: 437-41; and Stoll et al. (1988) Biomedical Chromatography 2(6): 249-253, each of which is hereby incorporated by reference in its entirety). The choice of specific binding pair members and the manipulation thereof to provide an interaction of the desired strength are well known to those of ordinary skill in this art.

In some embodiments, the multifunctional tag comprises a peptide substrate that is attached to a first nucleic acid such that hydrolysis of the peptide substrate provides a labeled hydrolytic product containing the first nucleic acid and a reporter but does not include the partitioner. In various aspects of this embodiment, the nucleic acid is a defined-sequence oligodeoxynucleotide, oligoribonucleotide, or a peptide nucleic acid. In such instances, a specific mobility-modifying polymer is non-covalently attached to the labeled hydrolytic product via a second nucleic acid covalently attached to the mobility modifier where the second nucleic acid is complementary to the first nucleic acid, wherein the hydrolytic product contains the first nucleic acid and a reporter but does not include the partitioner.

In some embodiments, where a plurality of proteases or protease families are to be detected, each of the multifunctional tags comprises a unique nucleic acid that hybridizes specifically to a particular mobility modifier that carries the complementary nucleic acid and which confers a distinct mobility upon the labeled hydrolytic product. The design and synthesis of such mobility modifiers comprising a nucleic acid, e.g. a peptide nucleic acid, that are useful for binding to a labeled hydrolysis product are readily adapted from those disclosed in U.S. Pat. No. 6,395,486 B1, which is hereby incorporated by reference in its entirety. In some embodiments, one or more reporter molecules are attached to the nucleic-acid-containing mobility modifier and the multifunctional tag may or may not have one or more reporter molecules attached thereto.

In certain embodiments, a multifunctional tag of the invention comprises a plurality of mobility-modifying polymers which are, collectively referred to herein, as the mobility modifier. In other embodiments, a multifunctional tag of the invention comprises a plurality of reporters.

3.3.3 Partitioner

The partitioner functions, in general, to separate unreacted, non-hydrolyzed multifunctional tags from the labeled hydrolytic products of interest that comprise a distinctive mobility modifier and a reporter and that are generated by hydrolysis of the multifunctional tags by the target proteases analyzed. The partitioner of a multifunctional tag is intended to possess traits that determine the net properties of the multifunctional tag. For example, where the multifunctional tag is intended to carry a net negative electrostatic charge, the partitioner comprises a sufficient number of acidic moieties such that the multifunctional tag carries a net negative electrostatic charge even though the mobility modifier, reporter, and peptide substrate carry a net positive electrostatic charge.

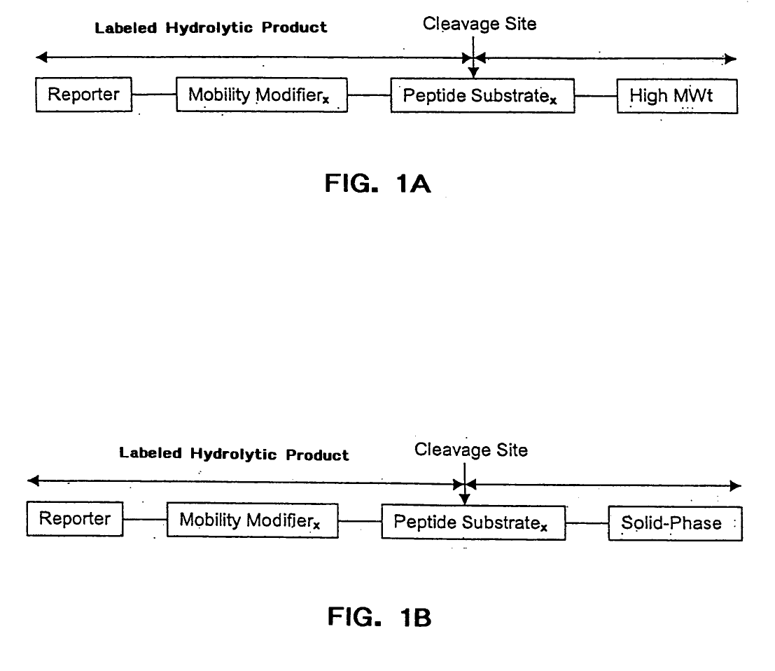

In some embodiments, the partitioner comprises a high molecular weight polymer of sufficient size that the corresponding non-hydrolyzed multifunctional tag, as well as any hydrolysis product thereof that includes the partitioner are readily separated by a chromatographic separation procedures that separate molecules according to size, from a product that comprises the mobility modifier and reporter, but not the partitioner, that is generated by proteolytic hydrolysis of that multifunctional tag. Such a multifunctional tag is depicted in FIG. 1A.

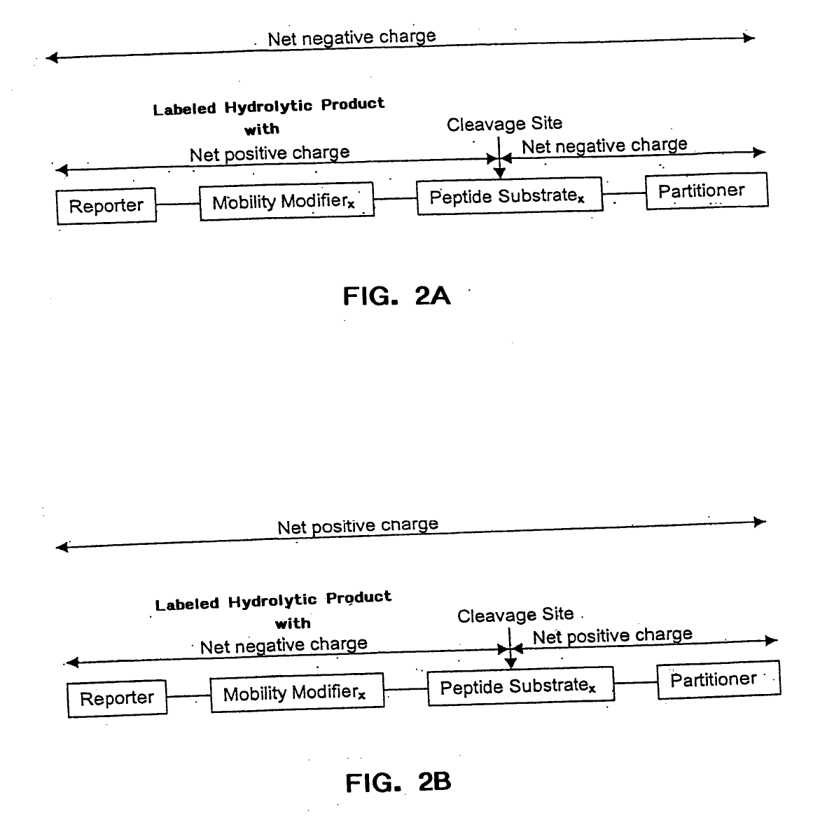

In some embodiments, the partitioner is positively charged such that a multifunctional tag comprising that partitioner carries a net positive electrostatic charge. In this embodiment, the mobility modifier, reporter and, in certain aspects of this embodiment, at least a portion of the peptide substrate attached to the mobility modifier, carry a net negative electrostatic charge, as depicted in FIG. 2B. Hydrolysis of the peptide substrate of the multifunctional tag by a target protease generates a negatively-charged hydrolysis product that comprises the reporter and mobility-modifier but does not include the partitioner, which is readily separated, e.g., by electrophoresis, from the positively-charged nonhydrolyzed multifunctional tag as well as any hydrolytic product of the multifunctional tag that comprises the partitioner.

In some embodiments, the partitioner is negatively charged such that a multifunctional tag comprising that partitioner carries a net negative charge. In this embodiment, the mobility modifier, reporter and at least a portion of the peptide substrate attached to the mobility modifier, carry a net positive charge. Hydrolysis of the peptide substrate of the multifunctional tag by a target protease provides a positively-charged hydrolysis product, which comprises the reporter and mobility-modifier but does not comprise the partitioner, that is readily separated, e.g. by electrophoresis, from the negatively-charged nonhydrolyzed multifunctional tag as well as any hydrolytic product of the multifunctional tag that comprises the partitioner. Such a multifunctional tag is depicted in FIG. 2A.

The partitioner, in some embodiments, comprises a polymer. Various polymers that can be adapted for use as a partitioner include, but are not limited to, polyethylene oxide, polyglycolic acid, polylactic acid, oligosaccharide, polyurethane, polyamide, polyimine, polyamine, polysulfonamide, polysulfoxide, and block copolymers thereof, including polymers composed of units of multiple subunits linked by charged or uncharged linking groups. Polymers useful as a partitioner to be included within the multifunctional tags of the present invention may be homopolymers, random copolymers, or block copolymers, either in a linear configuration, or, in certain embodiments, in a comb, branched, or dendritic configuration. Where the partitioner is a dendritic polymer, it may comprise a polyamidoamine branched polymer, which is commercially available from, e.g., Polysciences, Inc., Warrington, Pa.

In some embodiments, the partitioner is a polyoxide or polyether. In this context, the term polyoxide is used to denote polymers with oxygen atoms in the main chain, particularly those with monomer units of the type —[O—(CH₂)ₙ]— where n is an integer selected from the range of 1 to 15, in certain embodiments n is selected from the range of 2 to 6, and in other embodiments, n=2, together with their derivatives. Linear polyoxides useful as a partitioner include, for example, poly(methylene oxide), poly(ethylene oxide), poly(trimethylene oxide), poly(tetramethylene oxide), poly(pentamethylene oxide), poly(hexamethylene oxide), and poly(heptamethylene oxide). Branched polyoxides provide additional moieties available for partitioner-modification by, in some cases, imparting to multifunctional tag a molecular weight and size that facilitate separation of an unhydrolyzed multifunctional tag from a hydrolytic product thereof that comprises a reporter and a mobility-modifier but does not include a partitioner. Branched polymers, for example poly(propylene oxide) which are appreciably soluble in aqueous solvents, are used in certain embodiments. Other applicable branched polymers include poly(acetaldehyde), and poly(but-1-ene oxide).

In some embodiments, the partitioner comprises a polydisperse linear polyoxide of polyethyleneoxide (PEO) because of its high degree of solubility in a variety of aqueous and organic solvents. Where the partitioner is used as the basis for separating a multifunctional tag from a hydrolytic product thereof comprising a mobility modifier and reporter, the partitioner can have a nominal molecular weight of 1500 or more, 5000 or more, 10,000 or more, 20,000 or more, 50,000 or more, or 100,000 or more. Again, the chemistry of poly(ethylene oxide) and methods of use of such polymers for modifying chemical and biological compounds are well known in the art (see e.g. Grossman, P. D. et al., U.S. Pat. No. 5,777,096 and Lu et al. (1994) Int. J. Protein Res. 43: 127-138, both of which are hereby incorporated by reference in their entirety). Accordingly, those skilled in the art can readily vary the number of polyethylene units in a polymer to be used as a partitioner to impart a molecular weight, size, or charge that will facilitate separation of a multifunctional tag comprising that partitioner from a hydrolytic product thereof comprising a mobility-modifer and reporter but not including a partitioner.

Therefore, although substantially-monodisperse polymers can be used as partitioners in the assembly of the multifunctional tags of the present invention, in many embodiments, polydisperse polymer preparations can be used as a partitioner provided that substantially all members of that polydisperse population have a molecular weight and/or net charge that is sufficient to enable chromatographic and/or electrophoretic separation of the multifunctional tags from those hydrolytic products comprising a mobility-modifier and a reporter but not including a partitioner.

Moreover, particularly where a multifunctional tag is to be electrophoretically separated from the hydrolytic product thereof (which does not include the partitioner but does comprise the mobility modifier and reporter), the partitioner may comprise functional groups, such as carboxylate, phosphate, or secondary or tertiary amino moieties that will determine the net charge of both the partitioner as well as the net charge of the multifunctional tag that includes that partitioner.

In certain embodiments, the partitioner may comprise one or more ligands that are members of a binding pair, such as but are not limited to, molecules such as a low molecular weight antigen or hapten, e.g., cholesteryl, digoxigenin, or 2,4 dinitrophenol, or any other molecule, e.g. biotin, or a pheny boronic acid derivative that can be specifically and tightly bound by the second member of the binding pair.

Preferred polymers useful as a partitioner are those which ensure that the multifunctional tag is soluble in an aqueous medium, and which do not adversely affect hydrolysis of the peptide substrate by a target protease.

In some embodiments, the partitioner is an insoluble matrix or solid support to which the peptide substrate is attached, either directly or indirectly through a linker. Such an immobilized peptide substrate can be covalently or non-covalently attached to the solid surface either directly or indirectly through one or more linkers. In some embodiments, the mobility modifier and reporter are covalently attached, either directly or indirectly, to the peptide substrate and in such a manner that proteolytic hydrolysis of that peptide substrate releases a soluble product comprising the mobility-modifier and reporter. A multifunctional tag comprising a partitioner which is a solid surface is depicted in FIG. 1B.

In some embodiments, the partitioner is an agarose gel comprising activated hydroxyl groups that can be covalently conjugated with a primary amino group, such as the amino terminus of the peptide substrate or the side-chain amino group of lysine, or a sulfhydryl group, such as the side chain thiol moiety of cysteine. For example, beaded agarose is reacted with p-touenesulfonyl chloride (tosyl chloride) to provide an activated sulfonated support that will react with a nucleophile, particularly a sulfhydral group of a peptide substrate, to generate a disulfide bond joining the peptide substrate to the agarose matrix (see e.g. Nilsson et al. (1984) Methods in Enzymology 104: 56-69, which is hereby incorporated by reference). Similarly, agarose beads are activated with 1,4-butanediol diglycidyl ether, to provide an activated agarose derivative to which a primary amine of the substrate peptide can be covalently attached (see e.g. Sundberg et al. (1974) J. Chromatog. 90: 87-98, which is hereby incorporated by reference). Agarose beads are also reacted with 2,2,2-trifluoroethanesulfonyl chloride (tresyl chloride) (CF₃—CH₂—SO₂—Cl) to provide activated agarose beads which to which either a primary amine or sulfhydral moiety of the substrate peptide can be covalently attached (see e.g. Nilsson et al. (1984) Methods in Enzymology 104: 56-69). These activated agarose materials are commercially available from, e.g. Pierce, Rockford, Ill.

In some embodiments, the partitioner is an insoluble plastic matrix or solid plastic surface comprising active moieties to which a peptide substrate is covalently attached. In one illustrative, non-limiting, instance, the partitioner is a hexylamine-derivatized, nonporous spherical polystyrene bead. In this instance, a peptide substrate can be immobilized (1) by coupling a carboxyl moiety of the peptide substrate to the alkylamine bead in the presence of (1-ethyl-3[3-dimethylaminopropyl]carbodiimide hydrochloride (EDC) and N-hydroxysulfosuccinimide (SulfoNHS) (see e.g. Staros, J. V. (1986) Anal. Biochem. 156: 220-222); (2) coupling a sulfhydral group of the peptide substrate to the alkly amine in the presence of the heterobifunctional cross linker sulfosuccinimidyl 4-[N-maleimidomethyl]cyclohexane-1-carboxylate (Sulfo-SMCC) (see e.g. Hashida et al. (1984) J. Applied Biochem 6: 56-63); and (3) coupling an amino group of the substrate peptide to the alkyl amine in the presence of dimethylpimelimidate (DMP) (see e.g. Schneider et al. (1982) J. Biol. Chem. 257: 10766-69). Such alkyl-amine-activated plastic beads coupling reagents are commercially available from, e.g. Pierce, Rockford Ill.

In some embodiments, the partitioner comprises an insoluble matrix comprising crosslinked poly(ehtylene or propylene)glycol polymers as well as a spacer comprising functional groups to which the peptide substrate could be covalently attached. Beads formed of this material are readily derivatized using e.g. hydroxymethyl benzoic acid and an Fmoc-protected amino acid, and used for solid phase synthesis of a peptide. Moreover, such beads are sufficiently biocompatible and porous to allow enzymatic hydrolytic reactions to take place within the bead. One non-limiting example of such material is an insoluble matrix referred to as a “PEGA resin or polymer,” which is a polymer made up of approximately 60% O,O′-bis-(2-acrylamidoprop-1-yl)-PEG₉₀₀, about 20% O-(−2-acrylamidoprop-1-yl)-O′-(−2-aminoprop-1-yl)-PPG₃₀₀, and about 20% N,N-dimethyl acrylamide, where PEG₉₀₀ refers to a polyethylene glycol polymer of approximately 900 molecular weight (i.e. made up of approximately twenty ethylene glycol monomer units), and where PPG₃₀₀ refers to a polypropyleneglycol polymer of approximately 300 molecular weight (i.e. made up of approximately six to seven propylene glycol monomer units). In other embodiments, where the bead is to be porous to proteins of up to about 250,000 molecular weight, longer crosslinkers are used, e.g. PEG₄₀₀₀, PEG₆₀₀₀, and PEG₈₀₀₀. Such polymers and the methods and reagents for their synthesis are described in U.S. Pat. No. 5,352,756, and in Meldal et al. (1994) Proc. Natl. Acad. Sci. USA 91: 3314-18; Meldal, M. (2002) Biopolymers (Peptide Science) 66: 93-100; Meldal, M. (1998) Methods Mol. Biol. 87: 51-82.; and Meldal et al. (1998) J. Peptide Sci. 4: 83-91, each of which is hereby incorporated by reference in its entirety.

In some embodiments partitioner comprises an insoluble matrix or solid surface comprising the first member of a binding pair, while the second member of the binding pair is attached to the peptide substrate. In some embodiments, the first and second members of the binding pair are covalently attached to the insoluble matrix and peptide substrate, respectively. Illustrative non-covalent binding pairs include, but are not limited to, biotin and avidin; hapten (e.g cholesteryl, digoxigenin, or 2,4 dinitrophenol) and cognate antibody, antibody derivative (e.g. Fab fragment), or antibody-like molecule (e.g. single-chain antibody); phenyl boronic acid moiety and a salicylhydroxamic acid derivative; and a first nucleic acid and a second immobilized complementary nucleic acid to which the first nucleic acid can hybridize by Watson-Crick base-pairing or reverse Hoogstein base pairing.

In some embodiments, the insoluble matrix or solid surface is a glass or plastic surface to which one member of the binding pair is attached. In certain embodiments, one binding pair member is covalently attached to the surface. In one non-limiting illustration, the surface is a glass slide that is first silylated with an agent having the formula H₂N—(CH₂)ₙ—SiX₃ where n is between 1 and 10, and X is independently chosen from OMe, OEt, Cl, Br, and I, and then activated with a crosslinking reagent, followed by reacting with an amine-containing polymer. Silylating agents are chosen such that they react with the reactive groups present at the surface of the insoluble matrix or substrate to form a primary amine. For the purposes of the present illustration, the silylating agent is 3-aminopropyl-trimethoxysilane. The aminoalkylsilanated glass slide is treated with a multifunctional crosslinking reagent that comprises a reactive group at one end that can react with the nitrogen atom of an amine group to form a nitrogen-carbon bond. Such reactive groups are well known in the art, and include halides, esters, epoxides, and the like. The crosslinking agent additionally contains a protected reactive group at the opposite end that is capable of being deprotected and undergoing further reaction with an amine-containing polymer. Crosslinking reagents useful in this embodiment include, but are not limited to, N-succinimidyl-4-(iodoacetamido)-benzoate (SIAB), disuccinimidyl suberate, 1-ethyl-3-(dimethylaminopropyl)carbodiimide and 2,4,6-trichlorotriazine (cyanuric chloride). For the purposes of the present illustration, the crosslinking reagent is cyanuric chloride. The aminoalkylsilanated substrate treated with the crosslinking reagent may then be reacted with an amine-containing polymer. Any primary, secondary, or tertiary amine-containing polymer may be employed. The amine-containing polymer may be polyethylenimine, polyallylamine, polyvinylamine, and polyornithine. In the present illustration, the solid substrate, having been silylated and activated with the crosslinking reagent, is coated and modified with polyethylenimine (PEI). The PEI-coated glass slide is then used to immobilize polynucleotides, oligonucleotides, haptens, cytokines, proteins, peptides, saccharides, and the like. In some embodiments, the molecule to be immobilized is covalently attached via an alkylamino linker. Suitable methods and reagents that are adapted for use in this aspect of the invention are described in U.S. Pat. No. 6,387,631 B1, which is hereby incorporated by reference in is entirety.

In some embodiments, a hapten or other binding-pair member including but not limited to phenylboronic acid complexing reagents derived from aminosalicylic acid, avidin, or digoxigenin-binding antibody or derivative thereof, is attached to a partitioner that is an insoluble matrix or solid surface. For example, and only by way of illustration, phenylboronic acid complexing reagents are bound to an insoluble matrix or solid surface in various embodiments of the present invention. Suitable phenylboronic acid complexing reagents have been described in the art and include aminosalicylic acid derivatives comprising a linker which terminates in a carboxyl group as well as other such derivatives which terminate in a primary amino group (see e.g. U.S. Pat. No. 5,594,151, and U.S. Pat. No. 6,414,122 B1, each of which is hereby incorporated by reference in its entirety). Binding of such peptide substrates, haptens and other binding-pair members to the insoluble matrix or solid surface is mediated, as described above, by reactive chemical moieties that can be used to form a stable, preferably a covalent, bond with the molecule to be immobilized, either directly or via a multifunctional linker. The peptide substrates, haptens and other binding-pair members, insoluble matrix or solid surface are attached to one another using any chemically stable linkage, where the choice of linkage chemistry will depend on the nature of the mobility modifier, partitioner, reporter and amino acid moiety, hapten, or other binding-pair member. In some embodiments, the linkage is formed by the reaction of a primary or secondary amino moiety with a “complementary functionality.” For example, the complementary functionality can be isothiocyanate, isocyanate, acyl azide, N-hydroxysuccinimide (NHS) ester, sulfonyl chloride, aldehyde or glyoxal, epoxide, carbonate, aryl halide, imidoester, carbodiumide, anhydride, 4,6-dichlorotriazinylamine, or other active carboxylate (see e.g., U.S. Pat. No. 6,395,486 B1, and Hermanson, (1996) Bioconjugate Techniques, Academic Press, each of which is hereby incorporated by reference in its entirety). In some embodiments, the complementary functionality is an activated NHS ester which reacts with an amine. The activated NHS ester can be formed by reacting a carboxylate complementary functionality with dicyclohexylcarbodiimide and N-hydroxysuccinimide (see e.g. Khanna, et al. (1988) U.S. Pat. No. 4,318,846; and Kasai, et al., (1975) Anal. Chem., 47: 34037, each of which is hereby incorporated by reference in its entirety). In some embodiments, the partitioner is a glass surface derivatized with the phenylboronic acid complexing compound salicylhydroxamic acid, which is commercially available from Prolinx, Bothell, Wash.

3.3.4 Reporter

In some embodiments, the reporter, which is attached to the mobility-modifier-containing hydrolytic product of a peptide substrate, comprises a fluorescent dye that is used for detecting that mobility-modified product. In some embodiments, different dyes, which are preferably spectrally resolvable, are attached to different multifunctional tags in order to facilitate detection of the desired mobility-modified hydrolytic products generated by hydrolysis of a plurality of different peptide substrates by a plurality of target proteases in a multiplex assay. Fluorescent dyes useful as reporters include, but are not limited to, 5-carboxyfluorescein (5-FAM), 6-carboxyfluorescein (6-FAM), 2′,7′-dimethoxy-4′,5′-dichloro-6-carboxyfluorescein (JOE), N,N,N′-N-tetramethyl-6-carboxy rhodamine (TAMRA), 6-carboxy-X-rhodamine (ROX), 4,7,2′,4′,5′,7′-hexachloro-6-carbox-y-fluorescein (HEX-1), 4,7,2′,4′,5′,7′-hexachloro-5-carboxy-fluorescein (HEX-2), 2′,4′,5′,7′-tetrachloro-5-carboxy-fluorescein (ZOE), 4,7,2′,7′-tetrachloro-6-carboxy-fluorescein (TET-1), 1′,2′,7′,8′-dibenzo-4,7-dichloro-5-carboxyfluorescein (NAN-2), and 1′,2′,7′,8′-dibenzo-4,7-dichloro-6-carboxyfluorescein. Guidance for selecting appropriate fluorescent labels can be found in Smith et al. (1987) Meth. Enzymol. 155: 260-301, Karger et al. (1991) Nucl. Acids Res. 19: 4955-4962, Haugland (1989) Handbook of Fluorescent Probes and Research Chemicals (Molecular Probes, Inc., Eugene, Oreg.). Exemplary fluorescent labels include fluorescein and derivatives thereof, such as those disclosed in U.S. Pat. No. 4,318,846 to Khanna et al. and Lee et al. (1989) Cytometry 10: 151-164, U.S. Pat. No. 4,997,928 to Hobb, Jr., U.S. Pat. No. 4,855,225 to Fung et al., and PCT application US90/06608 to Menchen et al., each of which is hereby incorporated by reference in its entirety, and 6-FAM, JOE, TAMA, ROX, HEX-1, HEX-2, ZOE, TET-1 or NAN-2, as described above, and the like.

In some embodiments, where a plurality of fluorescent dyes are employed as reporters, they can be spectrally resolvable, such as, but not limited to the spectrally resolvable rhodamine dyes such as but not limited to those taught by Bergot et al. in PCT application PCT/US90/05565. In certain embodiments, the reporter is an energy-transfer dye pair, such as those disclosed in U.S. Pat. No. 6,465,645, which is hereby incorporated by reference in its entirety. In some embodiments the reporter includes two moieties, a fluorescent reporter and quencher, which together undergo fluorescence resonance energy transfer (FRET). The fluorescent reporter may be partially or significantly quenched by the quencher moiety in the intact peptide substrate of a multifunctional tag of the present invention. Hydrolysis of the peptide substrate of such a multifunctional tag releases a hydrolytic product comprising the mobility modifier and the fluorescent reporter but not the quencher nor the partitioner. In certain embodiments, a multifunctional tag comprises a plurality of reporters attached to the mobility-modifier and/or the peptide substrate such that hydrolysis of such multifunctional tags provides a hydrolytic product comprising a plurality of reporters and the mobility modifier but not the partitioner.

3.3.5 Linkers

Linkers may be used (1) to join monomer units to form a mobility modifier or a partitioner, (2) to join a reporter to a mobility modifier or to a peptide substrate, or (3) to join a peptide substrate to a mobility modifier or to a partitioner. Such joining can be accomplished using any chemically stable linkage, where the choice of linkage chemistry will depend on the nature of the involved moieties of the mobility modifier, partitioner, reporter and peptide substrate. In one embodiment, the linkage is formed by the reaction of a primary amino moiety, secondary amino moiety, hydroxyl group or sulfhydryl group, with a “complementary functionality.” Preferably, the complementary functionality is isothiocyanate, isocyanate, acyl azide, N-hydroxysuccinimide (NHS) ester, sulfonyl chloride, aldehyde or glyoxal, epoxide, carbonate, aryl halide, imidoester, carbodiumide, anhydride, 4,6-dichlorotriazinylamine, or other active carboxylate (see e.g., Hermanson, (1996) Bioconjugate Techniques, Academic Press). In some embodiments, the complementary functionality is an activated NHS ester which reacts with an amine, where the activated NHS ester is formed by reacting a carboxylate complementary functionality with dicyclohexylcarbodiimide and N-hydroxysuccinimide (Khanna, et al., (1988) U.S. Pat. No. 4,318,846; Kasai, et al., (1975) Anal. Chem., 47: 34037, each of which is hereby incorporated by reference in its entirety).

For example, fluorescent dyes used as reporters may include a reactive linking group at one of the substituent positions for covalent attachment of the dye to another molecule such as a mobility modifier or a peptide substrate. Reactive linking groups are moieties capable of forming a covalent bond and, typically include electrophilic functional groups capable of reacting with nucleophilic molecules, such as alcohols, alkoxides, amines, hydroxylamines, and thiols. Examples of such reactive linking groups include succinimidyl ester, isothiocyanate, sulfonyl chloride, sulfonate ester, silyl halide, 2,6-dichlorotriazinyl, pentafluorophenyl ester, phosphoramidite, maleimide, haloacetyl, epoxide, alkylhalide, allyl halide, aldehyde, ketone, acylazide, anhydride, and iodoacetamide. In some embodiments, the reactive linking group comprises a N-hydroxysuccinimidyl ester (NHS) of a carboxyl group substituent of a fluorescent dye. In some embodiments, the NHS ester form of the dye is used as the labeling reagent. The NHS ester of the dye may be preformed, isolated, purified, and/or characterized, or it may be formed and reacted with a nucleophilic group of a substrate, such as a mobility modifier or substrate peptide. The carboxyl form of a dye is activated by reacting with a carbodiimide reagent, e.g. dicyclohexylcarbodiimide, diisopropylcarbodiimide, or a uronium reagent, e.g. TSTU (O-(N-Succinimidyl)-N,N,N′,N′-tetramethyluronium tetrafluoroborate, HBTU (O-benzotriazol-1-yl)-N,N,N′,N′-tetramethyluronium hexafluorophosphate), or HATU (O-(7-azabenzotriazol-1-yl)-N,N,N′,N′-tetramethyluronium hexafluorophosphate), an activator, such as 1-hydroxybenzotriazole (HOBt), and N-hydroxysuccinimide to give the NHS ester of the dye. In some embodiments, a dye can be covalently bonded to the side-chain carboxyl moiety of aspartic or glutamic acid by direct coupling of an amino group of a dye with the side-chain carboxyl moiety using the activator BOP (Benzotriazol-1-yloxy-tris(dimethylamino)phosphonium hexafluorophosphate) to give an amide-bonded, peptide-dye conjugate. Other activating and coupling reagents include TBTU (2-(1H-benzotriazo-1-yl)-1-1,3,3-tetramethyluronium hexafluorophosphate), TFFH(N,N′,N″,N′″-tetramethyluronium 2-fluoro-hexafluorophosphate), PyBOP (benzotriazole-1-yl-oxy-tris-pyrrolidino-phosphonium hexafluorophosphate, EEDQ (2-ethoxy-1-ethoxycarbonyl-1,2-dihydro-quinoline), DCC (dicyclohexylcarbodiimide); DIPCDI (diisopropylcarbodiimide), MSNT (1-(mesitylene-2-sulfonyl)-3-nitro-1H-1,2,4-triazole, and aryl sulfonyl halides, e.g. triisopropylbenzenesulfonyl chloride. Where the dye contains a carboxyl group, the carboxyl may be activated, e.g. to the NHS for reaction with an amino moiety of a mobility modifier or of a side chain of an amino acid of a peptide substrate.

In some embodiments, reporters comprise, but are not limited to, radioisotopes, quantum dots, nanoparticles, lanthanide metals, and enzymes. In some embodiments, where a plurality of multifunctional tags are to be used, the same reporter is included within each of the multifunctional tags, while in other embodiments, one or more of the different multifunctional tags comprises a different reporter.

3.4 Multifunctional Tag Preparation

3.4.1 Peptide Substrate Synthesis

Peptide substrates to be incorporated within the multifunctional tag of the present invention can be synthesized by solid phase peptide synthesis (e.g., BOC or FMOC) methods, by solution phase synthesis, or by other suitable techniques including combinations of the foregoing methods. The BOC and FMOC methods, which are well established and widely used, are described in Merrifield (1963) J. Am. Chem. Soc. 88:2149; Meienhofer (1983) Hormonal Proteins and Peptides, C. H. Li, Ed., Academic Press, pp. 48-267; and Barany et al. (1980) in The Peptides, E. Gross and J. Meienhofer, Eds., Academic Press, New York, pp. 3-285. Solid phase peptide synthesis methods are also described in Merrifield, R. B., (1986) Science 232: 341; Carpino et al. (1972) J. Org. Chem. 37: 3404; and Gauspohl et al. (1992) Synthesis 5: 315 (1992)). Moreover, since its inception in 1962, R. B. Merrifield's basic method of solid phase peptide synthesis, now modified in a number of aspects, is an established technique in the art. Literally hundreds of investigations have been published describing the chemical details of the method (see e.g. Merrifield, R. B. (1965) Science 150: 178; Merrifield, R. B. (1968) Sci. Amer. 218: 56; Stewart et al. (1969) in Solid Phase Peptide Synthesis, San Francisco, Calif.: Freeman; Erickson et al. (1976) in The Proteins (eds. Neurath, R. L. Hill), III. Ed., Vol. 2, pp 255-527, New York: Academic Press); and Fields et al. (1990) Int. J. Peptide Protein Res. 35: 161-214. Each of the references cited in this paragraph are hereby incorporated by reference in its entirety.

In some embodiments, solid phase peptide synthesis begins with the covalent attachment of the carboxyl end of an (α-amino-protected) first amino acid in the peptide sequence, through a linker, to an insoluble resin bead (typically 25-300 microns in diameter). A general cycle of synthesis then consists of deprotection of the resin bound α-amino group, washing (and neutralization if necessary), followed by reaction with a carboxyl-activated derivative of the next (α-amino-protected) amino acid. These steps are repeated until the full-length peptide is synthesized. At the end of the synthesis, the peptide is cleaved from the solid support and purified. Automated instruments for solid phase synthesis of peptides have been described (see e.g. U.S. Pat. No. 5,186,898) and are commercially available, and include, e.g., the ABI 433A Peptide Synthesizer and the Pioneer™ Synthesis System, which allows the simultaneous synthesis of up to thirty-two different peptides, both of which are available from Applied Biosystems, Foster City, Calif. In addition, a high-throughput micromole-scale method for parallel synthesis and purification of peptides in a 96-well format has also been described (Pipkorn et al. (2002) J. Pep. Res. 59(3): 105-114, which is hereby incorporated by reference in its entirety).

In addition to the those amino acids constituting a protease-specific or substantially protease-specific peptide substrate for a target protease or target protease family, a peptide substrate may further comprise one or more amino acids, e.g. glycine, that provide structural flexibility or one or more amino acids, e.g. aspartic acid, glutamic acid, or lysine, that include a side chain used for attachment of, e.g. a reporter, mobility modifier, or partitioner. Such additional amino acids may also include amino acids, such as norleucine, citrulline and derivatives thereof, that are generally not found in proteins synthesized in vivo. The peptide substrate may also be synthesized to include one or more appended moieties as described below, that include but are not limited to one or more mobility modifiers, partitioners, reporters, linkers, and binding-pair members.

In some embodiments, the peptide substrate can be synthesized on a solid support that is a partitioner. The peptide substrate can be attached directly to, in one nonlimiting illustration, a PEGA resin or polymer as described above in Section 5.3.3. Cross-linked beads formed of this material are readily derivatized using e.g. hydroxymethyl benzoic acid and an Fmoc-protected amino acid and are used for solid phase synthesis of a peptide. In some embodiments, the peptide end directly attached to the insoluble matrix. In some embodiments, a linker, such as a peptide linker is attached directly to the insoluble matrix to obviate possible steric hindrance that could affect the rate of hydrolysis of the peptide substrate by a target protease.

In some embodiments, for example those in which the partitioner comprises a highly negatively-charged nucleic acid, the peptide substrate can be assembled on an immobilized nucleic acid bound e.g. to a controlled-pore-glass support. In some embodiments, the immobilized nucleic acid nucleic acid comprises a sequence-specific oligodeoxyribonucleotide member of a binding pair where the second member of the binding pair comprises a nucleic acid that comprises the complementary nucleotide sequence and is part of the partitioner, e.g. is covalently attached to an insoluble matrix or solid surface. In some embodiments, the immobilized nucleic acid can be a peptide nucleic acid that can be of high molecular weight and function as a partitioner in the methods and compositions of the present invention. Reagents, methods, and equipment used for the synthesis, and particularly the automated solid phase synthesis of nucleic acids, including oligodeoxyribonucleotides, and peptide nucleic acids, as well for the derivitization of such nucleic acids, e.g. by addition of a 5′-terminal linker comprising a terminal amino groups (such as but not limited to N-MMT-C6 Amino Modifier, which is a monomethoxytrityl protected amino linked phosphoramidite commercially available from e.g. Clontech Laboratories, Palo Alto, Calif.), upon which a peptide sequence can be assembled, are well known and widely used in the art (see e.g. U.S. Pat. No. 5,703,222, which is hereby incorporated by reference in its entirety).

In some embodiments, the peptide substrate can be assembled so as to incorporate one or more amino acids or amino acid analogues that have been derivatized to include a partitioner, mobility modifier, binding-pair member, or reporter. For example the peptide substrate can be synthesized to include the binding-pair compound biotin during solid phase peptide synthesis by using Fmoc-Lys(biotin)-OH (biotin is attached to the side chain ε-amino group of lysine by a peptide bond) which is commercially available from, e.g. Anaspec, San Jose, Calif. As with any of the binding-pair members or partitioners attached to the peptide substrate, the position of attachment is such that there is no substantial inhibition of hydrolysis of the peptide substrate by a target protease. Moreover, any binding-pair member, or partitioner attached thereto, is added such that hydrolysis of the peptide substrate by a target protease will release a product that comprises the mobility modifier or mobility modifiers and reporter or reporters, but does not include the partitioner or binding-pair member.

In some embodiments, the peptide substrate can be assembled so as to include an amino acid derivative comprising a phenylboronic acid moiety. For example, phenylboronic acid can be bound to the side chain amino group of lysine by conjugation with N-(3-dihydroxyborylphenyl)succinamic acid, succinimidyl ester, or to the side chain thiol of cysteine by conjugation with (3-maleimidophenyl)boronic acid using the method and reagents described in U.S. Pat. No. 5,494,111, which is hereby incorporated by reference in its entirety.

As described above, in some embodiments, the mobility modifier and the partitioner are, or comprise, polymers. More specifically, both the mobility modifier and partitioner comprise different derivatives of polyethylene glycol polymers, which have different properties that are exploited, as described above, to enable facile and substantially complete separation of labeled hydrolysis products comprising the mobility modifier(s) and reporter(s) from nonhydrolyzed multifunctional tags as well as hydrolysis products comprising the partitioner. In some embodiments, the mobility modifier(s) and/or partitioner(s) are covalently attached at defined positions to the peptide substrate during solid phase synthesis of the peptide substrate.

This is accomplished by incorporating one or more amino acid derivatives to which, e.g. an appropriate polyethylene glycol polymer has been attached. For example, reagents and methods have been described for the synthesis of two amino acid derivatives in which (1) a polyethylene glycol polymer has been covalently bonded to the α-amino group of norleucine or (2) to the side-chain amino group of ornithine. In addition, a third amino acid derivative was synthesized in which a polyethylene glycol polymer comprising an amino group was covalently attached to the carboxyl group of norleucine. In addition, an Fmoc-protected derivative of aspartic acid that carries a polyethyleneglycol polymer was synthesized by conjugating the side-chain carboxyl of Fmoc-protected aspartic acid with the amino group of the norleucine derivative carrying a polyethyleneglycol polymer bonded to the carboxyl group thereof. Similarly, an Fmoc-protected derivative of lysine carrying a polyethyleneglycol polymer was synthesized by conjugating the side-chain amino group of Fmoc-protected lysine with the carboxyl group of the norleucine derivative carrying a polyethyleneglycol polymer attached to the α-amino group thereof (see e.g. Lu et al. (1993) Peptide Research 6(3): 140-146; Lu et al. (1994) Int. J. Peptide Protein Res. 43: 127-138; and Campbell et al. (1997) J. Peptide Res. 49: 527-537, each of which is hereby incorporated by reference in its entirety). It is apparent that such methods and reagents are readily adapted to the direct labeling of the amino and carboxyl moieties of amino acids other than norleucine and ornithine with various polyethyleneglycol polymers comprising appropriate reactive moieties.

In some embodiments, amino acid derivatives carrying a polyethyleneglycol polymer bonded to the α-amino group can be incorporated into a growing peptide chain during solid phase synthesis of a peptide substrate, thereby attaching the polyethyleneglycol polymer, which in some embodiments can be either a mobility-modifier or a partitioner, to the amino terminus of the peptide substrate. Similarly, it is also apparent that amino acid derivatives carrying a polyethyleneglycol polymer bonded to a side-chain amino group of e.g. lysine or ornithine can be attached to the solid support used for the solid phase synthesis of a peptide substrate, thereby attaching a polyethyleneglycol polymer, which can be in some embodiments a mobility-modifier or a partitioner, to the carboxyl terminus of the peptide substrate.

Furthermore, in view of the ability to synthesize Fmoc-protected derivatives of e.g. aspartic acid and lysine which carry a polyethylene glycol polymer covalently bound to a side-chain carboxyl or amino moiety, it is apparent that one or more mobility modifiers or partitioners can be incorporated within the peptide substrate during solid phase synthesis thereof at positions other than the amino terminus and the carboxyl terminus.

In some embodiments, the peptide substrate can be synthesized to include one or more amino acids, e.g. cysteine or lysine, to provide a side-chain, reactive moiety that can be exploited for the covalent attachment of one or more binding pair members, dyes, mobility-modifiers, or partitioners. In each instance, the molecule or molecules to be added are joined directly to the peptide substrate or via a suitable linker.

3.4.2 Mobility Modifier Synthesis

Methods of preparing mobility-modifying polymer chains attached to the peptide substrate of the multifunctional tag generally follow known polymer subunit synthesis methods. These methods of forming selected-length polyethylene oxide-containing chains, which involve coupling of defined-size, multi-subunit polymer units to one another, directly or via linking groups, are applicable to a wide variety of polymers, such as polyethers (e.g., polyethylene oxide and polypropylene oxide), polyesters (e.g., polyglycolic acid, polylactic acid), oligosaccharides, polyurethanes, polyamides, polyamines, polysulfonamides, polysulfoxides, polyphosphonates, and block copolymers thereof, including polymers composed of units of multiple subunits linked by charged or uncharged linking groups. In addition to homopolymers, the mobility-modifying polymer chains used in accordance with the invention include selected-length copolymers, e.g., copolymers of polyethylene oxide units alternating with polypropylene units. For example, preparation of PEO chains having a selected number of HEO units involves protection of HEO at one end with dimethoxytrityl (DMT), and activation at its other end with methane sulfonate. The activated HEO can then react with a second DMT-protected HEO group to form a DMT-protected HEO dimer. This unit-addition is carried out successively until a desired PEO chain length is achieved.

Sequential coupling of HEO units can also be accomplished using uncharged bisurethane tolyl groups. Briefly, HEO is reacted with two equivalent of tolylene-2,4-diisocyanate under mild conditions, and the activated HEO is then coupled at both ends with HEO to form a bisurethane tolyl-linked trimer of HEO. Details of these two coupling methods are provided in U.S. Pat. No. 5,777,096, which is hereby incorporated by reference in its entirety.

Hydroxyl and carboxyl moieties of mobility-modifying polymers, as well as monomer units used to form mobility-modifying polymers, are readily activated using reagents and according to methods well known in the art (see e.g. EP 0 714 402 B9, U.S. Pat. No. 4,415,665, U.S. Pat. No. 5,470,705, U.S. Pat. No. 4,914,210, U.S. Pat. No. 5,777,096, U.S. Pat. No. 6,221,959 B1, and U.S. Patent Application Publication No. U.S. 2002/0182502 A1, each of which is hereby incorporated by reference in its entirety).

In some embodiments, the mobility modifier comprises a negatively-charged, polyanionic polymer comprising polyoxide monomeric units joined by phosphodiester bonds. For example, U.S. Pat. No. 5,777,096, which is hereby incorporated by reference in its entirety, describes the synthesis of DMT (dimethoxytrityl) protected hexaethyleneoxide phosphoramidite compounds. In some embodiments, a polyoxide, hexaethylene oxide (HEO) is reacted with dimethoxytrityl chloride. The desired mono-tritylated hexaethyleneoxide material (DMT-HEO) is isolated by silica gel chromatography and reacted with 2-cyanoethyl tetraisopropyl phosphordiamidite in the presence of tetrazole diisopropyl ammonium salt. The desired DMT-protected HEO phosphoramidite product (DMT-HEO-Phosphoramidite) is purified by flash chromatography through silica gel and eluted with 50% ethyl acetate/hexane (the silica gel was basified with triethylamine). Using standard phosphoramidite chemistry, DMT-HEO-Phosphoramidite can be conjugated with DMT-HEO to provide DMT-HEO-Phosphoramidite-HEO-DMT. Removal of the DMT protecting groups with weak acid provides the di-hydroxy compound HO-HEO-Phosphoramidite-HEO-OH. Deprotection of the phosphoramidite, e.g. using concentrated ammonia, provides the HO-HEO-(Phosphodiester)-HEO-OH product, which carries a negative charge at neutral pH. As is apparent to those skilled in the art, such methods and reagents can be used to generate a polyoxide having a defined number of ethylene oxide subunits with a defined number of anionic phosphodiester linking groups. As is also apparent, by varying the nature of the polyoxide monomer employed, as well as the nature of the linkage used in one or more condensations, a wide variety of polyoxide polymers of defined structure and charge can be generated readily. More particularly, the use of base-stable phosphotriester moieties as linking groups allows the inclusion of a defined number of uncharged phosphotriester linkages between selected polyoxide monomers (see e.g. U.S. Patent Application Publication No. U.S. 2002/0182602 A1, which is hereby incorporated by reference in its entirety).

Cationic polymers, in turn can, in one non-limiting illustration, be assembled using polyoxy monomers, that are conjugated, in a defined, stepwise manner, with e.g. alkyl amines to provide positively-charged copolymers and block copolymers using methods and reagents well known in the art (see e.g. U.S. Pat. No. 4,415,665 and U.S. Pat. No. 5,777,096, each of which is hereby incorporated by reference in its entirety). By way of illustration, mono-tritylated-hexaethylene glycol (DMT-HEO-OH) can be reacted with an alkyl sulfonyl halide such as methanesulfonylchloride to provide the activated DMT-HEO-sulfonyloxy derivative that is then coupled, in excess, with a compound comprising at least one primary or secondary amine moiety, e.g. ethylene diamine to provide in this non-limiting illustration the following positively charged compound which comprises two secondary amino groups: DMT-(OCH₂CH₂)₆CH₂CH₂NHCH₂CH₂NH(CH₂CH₂O)₆-DMT.

Therefore, it is apparent to those of ordinary skill that monodisperse and positively charged polycationic polymers can be readily assembled using reagents and methods well known in the art. Moreover, it is also apparent that such methods are readily adapted to the construction of a plurality of such monodisperse polymers differing, in a pre-determined manner, with respect to the number of monomer units and with respect to the number of cationic moieties included within such polymers that are useful as mobility-modifying polymers to be incorporated within the multifunctional tags of the present invention.

Also useful are mobility-modifying polymer chains which contain polyethylene oxide units linked by phosphoramidate linking groups, wherein aminoalkyl branching groups are attached to the phosphoramidate groups (Agrawal et al. (1990), Tetrahedron Letters 31(11): 1543-46). As noted above, the mobility-modifying polymer chain imparts to a hydrolytic product of each peptide substrate, an electrophoretic or chromatographic mobility which is distinctive for each different hydrolytic product. The contribution which the polymer chain makes to the mobility of each hydrolytic product will in general depend on the subunit length of the polymer chain. However, addition of charged groups to the polymer chain, such as charged linking groups in a polyethylene oxide chain, can also be used to achieve a selected mobility, e.g. a selected electrophoretic mobility, for a hydrolytic product of a peptide substrate.

3.4.3 Partitioner Synthesis

The partitioner confers properties upon the multifunctional tag within which the partitioner is incorporated that enable the facile and essentially complete separation of a labeled hydrolysis product, generated by proteolysis of the multifunctional tag by a target protease and which comprises the reporter and the multifunctional tag, from nonhydrolyzed multifunctional tags. Accordingly, a plurality or multiplicity of different multifunctional tags, each comprising a different peptide substrate and multifunctional tag which confers a unique mobility upon the labeled hydrolysis product generated from that multifunctional tag, may nevertheless all comprise the same partitioner. Thus, the partitioner facilitates the fractionation, detection, and identification of the labeled hydrolysis product without significant interference caused either by nonhydrolyzed multifunctional tags or by any hydrolytic product comprising the partitioner.

In certain embodiments, the partitioner is a polymer. In one aspect of this embodiment, where the analysis method used for fractionation of labeled hydrolysis products involves separations based upon size, the partitioner comprises a high molecular weight polymer of such a size that nonhydrolyzed multifunctional tags or any hydrolytic product thereof that comprise comprising the partitioner can be readily and essentially completely separated from a labeled hydrolytic product generated therefrom.

In other aspects of this embodiment, the partitioner is a polymer that carries a net electrostatic charge large enough to confer the same net type of charge upon the multifunctional tag within which the partitioner is incorporated. That is, where the partitioner is an anionic polymer, the corresponding multifunctional tag is negatively charged and where partitioner is a positively-charged cationic polymer, the corresponding multifunctional tag is positively charged.

In the former instance in which the partitioner and multifunctional tag are anionic, the mobility modifier is designed so that the labeled hydrolytic product generated from that multifunctional tag is a cation. Similarly, in instance in which the partitioner and multifunctional tag are cationic, the mobility modifier is designed so that the labeled hydrolytic product generated from that multifunctional tag is an anion. Accordingly, in each instance nonhydrolyzed multifunctional tags and hydrolytic products comprising the partitioner are readily and essentially completely separated from labeled hydrolytic products comprising the reporter and mobility modifier.

Therefore, in some embodiments, the partitioner comprises a hydrophilic, relatively high molecular weight polymer. Such polymers, which are readily prepared using reagents and methods well known in the art, comprise polyethylene oxide, polyglycolic acid, polylactic acid, oligosaccharide, polyurethane, polyamide, polyamine, polyimine, polysulfonamide, and polysulfoxide polymers, as well as block copolymers thereof, including polymers composed of units of multiple subunits linked by charged or uncharged linking group. In some embodiments, the partitioner is a nucleic acid, e.g., an oligodeoxyribonucleotide.

In some embodiments, the polymer can be synthesized, by way of illustration and not limitation, by conjugation of hexaethyleneoxide monomers to provide higher molecular weight structures, which may be cationic, anionic, or neutral polymers, using reagents and methods disclosed in U.S. Pat. No. 5,777,096, U.S. Pat. No. 5,703,222, U.S. Pat. No. 6,221,929, and U.S. Pat. No. 4,415,665, each of which is hereby incorporated by reference in its entirety.

In some embodiments, an uncharged, electrostatically-neutral polyoxide polymer is assembled by repeated condensation of, e.g., hexaethylene oxide units (HEO). Hexaethylene glycol is reacted with dimethoxytrityl chloride and the crude product purified by silica gel chromatography to provide mono-tritylated HEO (DMT-HEO). The DMT-protected HEO is reacted with methanesulfonyl chloride in the presence of diisopropylethylamine to provide the DMT-protected HEO-mesylate. An HEO dimer is synthesized by mixing hexaethylene glycol into a suspension of sodium hydride followed by reaction with DMT-protected HEO-mesylate. The DMT-protected product, now comprising twelve ethylene oxide units, is purified by silica gel chromatography. Repetition of these step therefore provides nonionic, longer HEO polymer chains of increasing molecular weight.

In some embodiments, an anionic polyoxide polymer is assembled by repeated condensation of, e.g., hexaethylene oxide units (HEO) in which individual units are joined by phosphodiester bonds. In this illustration, hexaethylene glycol is reacted with dimethoxytrityl chloride and the crude product purified by silica gel chromatography to provide mono-tritylated HEO (DMT-HEO). The DMT-protected HEO is reacted with 2-cyanoethyl tetraisopropyl phosphordiamidite in the presence of tetrazole diisopropyl ammonium salt to provide, after silica gel chromatography, DMT-protected HEO phosphoramidite. Mono-trityl-protected HEO is then reacted with the DMT-protected HEO phosphoramidite generally according to phosphoramidite chemistry methods to provide a DMT-protected product comprising two HEO monomer blocks linked by a cyano-ethyl phosphate triester linkage group. Removal of the DMT groups provides a diol derivative that is reacted with two equivalents of DMT-protected HEO phosphoramidite to provide a product comprising a total of four HEO monomers joined by a cyano-ethyl phosphate triester linkage group. These procedures can be repeated until an HEO polymer of the desired size is achieved. Removal of the cyanoethyl groups with mild acid provides an HEO polymer in which the individual monomers are linked with negatively charged phosphodiester groups.

In a some embodiments, a positively charged, cationic polyoxide polymer is assembled by repeated condensation of, e.g., hexaethylene oxide units (HEO) along with e.g. ethylene diamine. Hexaethylene glycol is reacted with dimethoxytrityl chloride and the crude product purified by silica gel chromatography to provide mono-tritylated HEO (DMT-HEO). The DMT-protected HEO is reacted with methanesulfonyl chloride in the presence of diisopropylethylamine to provide the DMT-protected HEO-mesylate. Conjugation of two equivalents of DMT-protected HEO-mesylate with ethylene diamine therefore would provide a DMT-protected linear product comprising one ethyl monomer joined at each end via an amino linking group to a DMT-protected HEO monomer. Moreover, substitution of N-(3-aminopropyl)-1,3-propane diamine for ethylene diamine in this synthesis procedure provides a branched structure comprising three HEO monomers joined to each of the nitrogen atoms of the N-(3-aminopropyl)-1,3-propane diamine monomer, and, therefore, including both secondary and tertiary amino moieties.

Although the synthesis of each of the partitioner polymers is illustrated with reactions involving the joining of defined monomers to provide defined polymers, that would be expected to be substantially monodisperse, it is apparent that a polydisperse polymer can be used as a partitioner. Accordingly, the above syntheses, which are only illustrative of the methods available, could be carried out substituting one or more commercially-available compounds for the reagents provided. For example, various polyethylene and polypropylene materials, having a molecular weight of 1000 to 40,000, are available from, inter alia, Shearwater Corporation (Huntsville, Ala.) as: (1) diols having a nominal molecular weight of 20,000, (2) branched chain polyols having up to eight “arms” having a nominal molecular weight of up to 40,000, (3) activated derivatives including succinamide, benzotriazole, and aldehyde moieties that can react with amino groups to generate amide, carbamate, and secondary amine linkages, respectively, (4) an ethyleneglycol derivative comprising a covalently-bound biotin moiety as well as an activated (NHS) carboxy group, and (5) ethylene glycol derivatives comprising an FMOC-protected primary amino group as well as an N-hydroxysuccinamide (NHS)-protected carboxyl group which can be assembled into polymers in which the monomeric units are joined by peptide bonds, essentially using standard peptide-synthesis chemistry, and could be carried out on a solid phase, automated peptide-synthesis instrument.

Moreover, the approaches described above for synthesis of a polymer-containing partitioner could also be used for the construction of a plurality different polymer blocks that can be joined in the construction of a block copolymer comprising, as but one example, a combination of both cationic and uncharged, electrostatically neutral polymer blocks.

In some embodiments, the partitioner comprises a solid substrate or a matrix to which the peptide substrate is attached either directly or through a linker. In such embodiments, the mobility modifier and the reporter are also joined, directly or through a linker, to the peptide substrate. The peptide bond of the peptide substrate that is hydrolyzed by the target protease is disposed between the partitioner and the mobility modifier/reporter. Accordingly, proteolytic hydrolysis of the peptide substrate releases a labeled hydrolytic product comprising the mobility modifier and the reporter.

In some embodiments, the peptide substrate is linked either covalently or non-covalently to the solid surface or matrix, as described in Section 3.4.4, below.

3.4.4 Assembly of the Multifunctional Tag

Coupling of a mobility modifying polymer and/or a partitioner to a peptide can be carried out by an extension of conventional peptide synthesis methods, or by other standard coupling methods. That is, e.g., a polymeric mobility modifier can be built up on a peptide substrate by stepwise addition of mobility-modifying polymer-chain units to the peptide substrate, using standard solid-phase synthesis methods. Stepwise addition of e.g. hexaethylene oxide units, which comprise a carboxy moiety at one end and an amino group at the other, to an immobilized substrate peptide, via amide linkages is accomplished using chemistry that is similar to or readily adapted from that used in conventional peptide synthesis.

In some embodiments, the mobility modifier can be covalently attached to the amino terminus of the substrate peptide while the partitioner comprises a polymer covalently attached to the carboxyl terminus of the peptide substrate. Alternatively, the mobility modifier can be covalently attached to the carboxyl terminus of the substrate peptide while the partitioner comprises a polymer covalently attached to the amino terminus of the peptide substrate. In some embodiments the mobility modifier and/or the partitioner comprises a polymer attached to the side chain of an amino acid of a peptide substrate, where that amino acid is neither the amino-terminal nor the carboxyl-terminal residue of the peptide substrate.

In some embodiments, the peptide substrate may be covalently labeled by conjugation with a sulfonated dye. For example, the dye is in electrophilic form, e.g. comprises an NHS reactive linking group, which reacts with a nucleophilic group of the peptide, e.g. an amino side chain of an amino acid such as lysine. Alternatively, the dye may be in nucleophilic form, e.g. amino- or thiol-reactive linking group, which may react with an electrophilic group of the peptide, e.g. NHS of the carboxyl side chain of an amino acid. Peptide substrates can also be labeled with two moieties, a fluorescent reporter and quencher, which together undergo fluorescence resonance energy transfer (FRET). The fluorescent reporter may be partially or significantly quenched by the quencher moiety in an intact peptide. Upon cleavage of the peptide by a protease, a detectable increase in fluorescence may be measured (Knight, C. (1995) “Fluorimetric Assays of Proteolytic Enzymes,” Methods in Enzymology, Academic Press, 248: 18-34). A general protocol for conjugating the dyes in the NHS ester form to peptide substrates entails dissolving the NHS esters in aqueous acetonitrile (the percentage of acetonitrile is determined by the hydrophobicity of the dye to attain solubility) with peptides in water (or aqueous acetonitrile solution if peptides were hydrophobic). Aqueous sodium bicarbonate buffer (1 M) is added to the solution to achieve 0.1 M buffer concentration while vortexing or shaking. The mixture is shaken at room temperature for 10 minutes to 30 minutes. The crude peptide-dye conjugate in the reaction mixture can be directly purified by reverse-phase HPLC, to provide the desired dye-labeled peptide.

3.5 Separation and Detection of Labeled Hydrolytic Products

In some embodiments, a plurality of different multifunctional tags are contacted with a sample comprising a plurality of proteases, where the different multifunctional tags comprise a different peptide substrate that is specific or substantially specific for a target protease or protease family. Moreover, in this embodiment each different peptide substrate is attached to a particular mobility modifier and, directly or indirectly to one or more reporters, such that hydrolysis of the peptide substrate generates a hydrolytic product that does not include the partitioner but comprises one or more reporters and a mobility modifier that confers a distinct mobility, e.g. a distinct electrophoretic mobility, upon the labeled hydrolytic product.

Therefore, in some embodiments, different hydrolytic products of a peptide substrate, which by themselves are difficult to resolve by chromatographic or electrophoretic methods, can be finely resolved using e.g. a mobility-dependent analysis technique via the mobility-modifying polymer or moieties attached to the peptide substrate. The method is particularly useful in resolving hydrolytic products of multifunctional tags that comprise peptide substrates of substantially the same length and/or charge.

3.5.1 Separation of Labeled Hydrolytic Products Comprising a Mobility Modifier and a Label by Chromatography

In some embodiments, labeled hydrolytic products, which comprise a reporter and mobility modifier, but do not comprise a partitioner, that are generated by hydrolysis of the peptide substrate of a multifunctional tag by a target protease or target protease family, are resolved (separated) by liquid chromatography. Exemplary solid phase media for use in the method include reversed-phase media (e.g., C-18 or C-8 solid phases), ion exchange media (e.g. cation and anion exchange media), and hydrophobic interaction media. In some embodiments, the labeled hydrolytic products can be separated by micellar electrokinetic capillary chromatography (MECC).

Reversed-phase chromatography is carried out using an isocratic, or more typically, a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. For separating labeled hydrolytic products comprising charged moieties, an ion pairing agent, e.g., a tetraalkylammonium species, is typically included in the solvent to mask the charge of e.g. phosphate oxyanions.

The mobility of the labeled hydrolytic products can be varied by addition of polymer chains that alter the affinity of the probe for the solid phase. Thus, with reversed phase chromatography, an increased affinity of the labeled hydrolytic product for the solid phase can be attained by attaching a moderately hydrophobic polymer (e.g., PEO-containing polymers, short polypeptides, and the like) to the peptide substrate. Longer attached polymers impart greater affinity for the solid phase, and thus require higher non-polar solvent concentration for the labeled hydrolytic product to be eluted (and a longer elution time). In such instances the partitioner can be, e.g., an insoluble matrix or surface.

Generally, in anion exchange chromatography, charged analytes are eluted from an oppositely-charged solid phase using a salt gradient, where analytes elute according to the number and distribution of charges in each analyte. For example, where the labeled hydrolytic product is a polyanion, hydrolytic products of essentially the same size elute generally according to the net charge of the hydrolytic product, with the least charged hydrolytic products eluting first, with more highly-charged hydrolytic products eluting later as the concentration of salt is increased over time. Thus, where anion exchange chromatography is used in the method of the invention, the mobility modifiers attached to the peptide substrates may comprise positively charged polymer chains or moieties in order to reduce the affinity of a labeled hydrolytic product for the solid phase, and negatively charged polymers and moieties can be included in the mobility modifier to increase affinity for the solid phase. Similar considerations apply to hydrophobic interaction chromatography.

In micellar electrokinetic capillary chromatography (MECC), different labeled hydrolytic products are separated by electrophoretic passage through a separation medium that contains micelles formed by surfactant molecules (e.g., sodium dodecyl sulfate). Sample separation is mediated by partitioning of the sample components between a primary phase, formed by the running buffer, and a secondary phase, formed by micelles, in a separation process that may be characterized as a form of chromatography. For enhanced separation of the labeled hydrolytic products, the separation medium may contain divalent metal ions, for complexing with anionic moieties of e.g. a mobility modifier to modify their mobility (see e.g. Grossman, P. G. and Colburn, J. C. Eds. Capillary Electrophoresis, Academic Press, Inc., San Diego, Calif. (1992); Cohen et al. (1987) Anal. Chem. 59(7): 1021).

3.5.2 Separation of Labeled Hydrolysis Products by Electrophoresis in a Sieving Matrix

In some embodiments, labeled hydrolytic products can be resolved by electrophoresis in a sieving matrix. For example, the electrophoretic separation is carried out in a capillary tube. Sieving matrices which can be used include covalently crosslinked matrices, such as acrylamide covalently crosslinked with bis-acrylamide (Cohen et al. (1990) J. Chromat. 516: 49); gel matrices formed with linear polymers (Matthies et al. (1992) Nature 359: 167); and gel-free sieving media (U.S. Pat. No. 5,089,111 to Zhu et al. (1992)), for example. The percentage of acrylamide in polyacrylamide-containing matrices can range from about 3.5% to about 20% for achieving the desired separation of a plurality of labeled hydrolytic products generated in the methods of the invention. The electrophoresis medium may also contain a denaturant, such as 7M formamide or 8M urea, for maintaining polymers of the mobility-modifier, e.g. in single an extended conformation and to minimize interactions between and among mobility-modifying polymers where necessary.

Within a sieving matrix, the mobility of each labeled hydrolytic product depends on net charge and on size. For example, smaller more highly negatively charged labeled hydrolytic products migrating more rapidly than larger, less-highly negatively charged labeled hydrolytic products. Thus, where different labeled hydrolytic products both carry a similar net charge, essentially any polymer chain can be used to impart lower mobility on a given labeled hydrolytic product, by increasing the overall size of the product to which the polymer chain is attached. In some embodiments, therefore, the attached polymer chains are uncharged, while in other embodiments, the mobility modifying polymer chain can carry one or more positively-charged moieties in order to reduce the mobility of a given labeled hydrolytic product relative to that of another labeled hydrolytic product carrying a greater net negative charge, since the greater net negative charge provides a greater net electrical force that is effective to draw the probe through the electrophoretic medium.

3.5.3 Separation of Labeled Hydrolysis Products by Electrophoresis in a Non-Sieving Matrix

In some embodiments, labeled hydrolysis products, which comprise a mobility modifier and a reporter but not a partitioner, are fractionated by capillary electrophoresis in a non-sieving matrix, as defined above. The advantage of capillary electrophoresis is that efficient heat dissipation reduces or substantially eliminates thermal convection within the medium, thus improving the resolution obtainable by electrophoresis.

Electrophoresis, such as capillary electrophoresis, (CE) can be carried out by standard methods, and using conventional CE equipment, except that the electrophoresis medium itself does not contain a sieving matrix.

The ability to fractionate labeled hydrolysis products by electrophoresis in the absence of a sieving matrix offers a number of advantages. One of these is the ability to fractionate a plurality of hydrolytic products of different peptide substrates that may be of about the same size and have about the same net charge via attachment of a mobility modifier, which may comprise one or more polymer chains, which imparts a unique charge to translational frictional drag ratio to the hydrolytic product to which it is attached. As will be appreciated, this feature allows the peptide substrates in the multifunctional tag compositions to have similar sizes, and/or net electrostatic charges, and thus similar physical properties. Another advantage is the greater convenience of electrophoresis, particularly CE, where sieving polymers and particularly problems of forming and removing crosslinked gels in a capillary tube are avoided.

3.5.4 Detection of Labeled Hydrolytic Products

For detection purposes, the labeled hydrolytic products of the invention contain, or can be modified to contain, a reporter that allows direct detection of a labeled hydrolytic product by a suitable detector.

In some embodiments, the reporter comprises a fluorescent label which is spectrally resolvable as defined above in Section 5.1. For example, one or more reporters may be attached directly or via a linker to the peptide substrate and/or the mobility modifier by methods known in or adapted from the art (see e.g. Fung et al., U.S. Pat. No. 4,855,225; Prober et al (1987) Science 238: 4767-4771; Smith et al. (1985) Nucleic Acids Res. 13: 2399-2412; and Lee et al. U.S. Pat. No. 6,372,907; and the like, each of which is hereby incorporated by reference in its entirety).

Exemplary dyes which can be used as a reporter include but are not limited to 5- and 6-carboxyfluorescein, 5- and 6-carboxy-4,7-dichlorofluorescein, 2′,7′-dimethoxy-5- and 6-carboxy-4,7-dichlorofluorescein, 2′,7′-dimethoxy-4′,5′-dichloro-5- and 6-carboxyfluorescein, 2′,7′-dimethoxy-4′,5′-dichloro-5- and 6-carboxy-4,7-dichlorofluorescein, 1′,2′,7′,8′-dibenzo-5- and 6-carboxy-4,7-dichlorofluorescein, 1′,2′,7′,8′-dibenzo-4′,5′-dichloro-5- and 6-carboxy-4,7-dichlorofluorescein, 2′,7′-dichloro-5- and 6-carboxy-4,7-dichlorofluorescein, and 2′,4′,5′,7′-tetrachloro-5- and 6-carboxy-4,7-dichlorofluorescein. The above-mentioned dyes are disclosed in the following references, each of which is hereby incorporated by reference in its entirety: Hobb, Jr. U.S. Pat. No. 4,997,928; Fung et al, U.S. Pat. No. 4,855,225; and Menchen et al, PCT application no. PCT/US90/06608. In some embodiments, probes may be labeled with spectrally resolvable rhodamine dyes such as but not limited to those taught by Bergot et al, PCT application no. PCT/US90/05565, and by Lee et al. U.S. Pat. No. 6,372,907.

In some embodiments, the labeled hydrolytic products are resolved by electrophoresis in a sieving or non-sieving matrix. In some embodiments, the electrophoretic separation is carried out in a capillary tube by capillary electrophoresis (e.g., Capillary Electrophoresis: Theory and Practice, Grossman and Colburn eds., Academic Press (1992)). Sieving matrices include, but are not limited to, covalently crosslinked matrices, such as polyacrylamide covalently crosslinked with bis-acrylamide; gel matrices formed with linear polymers (e.g., Madabhushi et al. U.S. Pat. No. 5,552,028); and gel-free sieving media (e.g., Grossman et al., U.S. Pat. No. 5,624,800; Hubert et al. (1995) Electrophoresis, 16: 2137-2142; Mayer et al. (1994) Analytical Chemistry, 66(10): 1777-1780). Suitable capillary electrophoresis instruments are commercially available, e.g., the ABI PRISM™ Genetic Analyzer (PE Biosystems, Foster City, Calif.).

3.6 Assay Methods and Embodiments

In some embodiments, there are provided compositions and methods that can be used for detection of hydrolytic enzymes, in particular, to proteolytic enzymes, i.e. proteases and peptidases (which terms are used interchangeably). In some embodiments, methods are provided for detection and/or quantitation of one or more proteases in sample. In some embodiments, a protease-containing sample may comprise one or a plurality of purified proteases. In some embodiments, a protease-containing sample may comprise one or more substantially unpurified proteases e.g., in one non-limiting example, a crude extract prepared from a recombinant organism that has been genetically engineered to overexpress one or more target proteases. In some embodiments, the protease-containing sample comprises a substantially-unfractionated extract prepared from a tissue or from a cell line.

Some embodiments provide methods and compositions for the construction and use of multifunctional tags in the detection and/or quantitation of one or more proteases. Such multifunctional, in some embodiments, comprise a reporter, mobility modifier, peptide substrate, and a partitioner. The peptide substrate can be specific or substantially specific or a single protease or protease family. Design and selection of peptide substrates useful for incorporation into the multifunctional tags of the invention, can be carried out using one or more of the approaches described above in Section 5.2.1.

Hydrolysis of a multifunctional tag of the invention by a cognate protease provides a labeled hydrolytic product comprising a reporter and a mobility modifier but does not include a partitioner. In some embodiments, a protease-containing sample comprising a plurality of proteases is contacted with a composition comprising a plurality of different multifunctional tags. Each multifunctional tag of the plurality comprises a different peptide substrate that is specifically or substantially specifically hydrolyzable by a particular protease or protease family member to generate a labeled hydrolytic product. Moreover, each different peptide substrate is associated with a different mobility modifier which provides the corresponding labeled hydrolytic product with a distinctive mobility in a mobility-dependent analysis method, e.g. a distinctive electrophoretic mobility.

Where a plurality of such labeled hydrolytic products are generated by contacting a protease-containing sample with a plurality of multifunctional tags of the invention, those labeled hydrolytic products are separated using a mobility-dependent analysis method. In some embodiments the mobility-dependent analytical method is an electrophoretic method, such as, but not limited to capillary electrophoresis, which can be carried out in either a sieving or a non-sieving medium. In the latter instance, the mobility modifier of each different multifunctional tag provides the corresponding labeled hydrolytic product with a distinctive ratio of charge to translational frictional drag enabling the plurality of labeled hydrolytic products to be resolved from one another.

In some embodiments, the reporter of the multifunctional tag comprises a fluorescent dye having an absorption spectrum that encompasses the output of a laser light source. Accordingly, in such embodiments, a plurality of labeled hydrolytic products can be resolved by electrophoresis. In some embodiments, the electrophoresis capillary electrophoresis that is carried out in a sieving medium. In some embodiments, the electrophoresis is capillary electrophoresis that is carried out in a non-sieving medium and each labeled hydrolytic product is detected by laser-induced fluorescence as that labeled hydrolytic product passes the detection window of the analysis instrument used. Such equipment is well-known in the art and is commercially available, e.g. ABI PRISM™ Model 3700 Genetic Analyzer (PE Biosystems, Foster City, Calif.).

In some embodiments, the above hydrolytic reactions are performed under conditions established so that the amount of labeled hydrolysis product detected may be directly proportional to the amount of a target protease present in the protease-containing sample tested. That is, the rate of such hydrolytic reactions may be linear with respect to time and with respect to the amount of protease-containing sample used. Such conditions, as well as, in various aspects of this embodiment, the inclusion of one or more standards in the hydrolysis reactions and/or the separation method, facilitate such quantitation.

3.6.1 Therapeutic Target Discovery

In some embodiments, multifunctional tags are used to identify hydrolytic enzymes, particularly proteases, that have different levels of catalytic activity in normal tissue as compared with the corresponding diseased tissue. Such enzymes therefore could be predicted to be involved in the onset, development, and/or progression of the particular disease examined. Consequently, proteases identified in this manner could be potential targets for effective therapeutic intervention for the treatment or prevention of that disease. In some embodiments, the disease is a particular form of cancer and the diseased tissue is a pre-neoplastic tissue, an invasive cancer tissue, or a metastatic cancer tissue. In some embodiments, the diseased tissue corresponds to that involved in rheumatoid arthritis or muscular dystrophy. In some embodiments, the diseased tissue is a tissue infected with, in non-limiting examples, a virus, such as the HIV virus, or a pathogenic microorganism such as a bacterium, fungus, or parasitic agent.

In some embodiments, extracts prepared from both normal tissue and the corresponding diseased tissue are contacted with a composition comprising a plurality of different multifunctional tags. In this instance, the plurality of multifunctional tags includes a number of different peptide substrates that are known to be specifically or substantially specifically hydrolyzed by a particular protease or protease family. Accordingly, the presence of a defined labeled hydrolytic product, as well as the amount thereof, is indicative of the presence and amount of a particular protease or protease family in the tissue sample examined. In some embodiments, the extracts to be compared are each contacted with a composition comprising a different set of a plurality of multifunctional tags and the labeled hydrolytic products generated in each instance are combined prior to their separation and detection. The sets differ only with respect to the reporter of the multifunctional tag and, in certain embodiments, the different reporters are spectrally-resolvable fluorescent dyes. Accordingly, differing levels of particular proteases are indicated by comparing peak heights at each mobility address. Where a particular mobility address has been determined to correspond to the known hydrolytic product, then it can be directly established which protease or protease family differs in its level of catalytic activity between the samples examined.

3.6.2 Diagnostic Methods

In some embodiments, one or more peptide substrates are identified that are specifically or substantially specifically hydrolyzed by either normal tissue or a particular diseased tissue, generally according to the methods of Section 3.2.1. In some embodiments, phage-display methods are used to identify those peptides that are efficiently hydrolyzed by proteases present in extracts prepared from diseased tissue but not in those from normal tissue. In a similar manner, peptides are identified that are efficiently hydrolyzed by proteases present in extracts of normal tissue but not in those from diseased tissue. In some embodiments, a population of phage is first enriched for those displaying peptides effectively cleaved by proteases present in extracts of one tissue type and then depleted of those phage displaying peptides effectively cleaved by proteases present in extracts of the the alternative tissue type, according to methods described in Section 5.2.1.4, above. Such cycles of enrichment+depletion are repeated until an appropriate number of peptide sequences are identified that can be used to develop a “fingerprint” of protease activity that is diagnostic of each tissue type to be compared.

Similarly, other methods, such as those described in Section 3.2.1.3, can also be adapted for the identification of peptide substrates that are specifically or substantially specifically hydrolyzed by proteases present in diseased tissue or present in normal tissue. For example, a library of non-fluorescent (FRET) peptides is first contacted with an extract prepared from normal tissue cells and the fluorescent beads, which comprise peptide sequences readily cleaved in normal extracts, are removed. The resulting library, which has been “depleted” of sequences cleaved in normal tissue, is then contacted with an extract prepared from a diseased or infected tissue to discern those peptide substrates that are readily cleaved in the diseased tissue but not the normal tissue. By reversing the order of the reactions, it is also possible to discover those peptide substrates cleaved in normal tissue but not in diseased tissue. In some embodiments, it is not essential that the identity of the particular protease, proteases, protease family, or protease families present in the extract tested be determined.

Such peptides that are cleaved with different hydrolytic efficiency by extracts from normal tissue as compared extracts from diseased tissue are used to construct a series of different multifunctional tags. Each different multifunctional tag comprises one of the particular peptide substrates identified as an indicator of diseased or normal tissue and a different mobility modifier. In some embodiments, two spectrally-resolvable reporters are used with one incorporated within multifunctional tags preferentially cleaved by proteases present in extracts from diseased tissue and the other incorporated within multifunctional tags preferentially cleaved by proteases present in extracts from normal tissue. Use of different, spectrally resolvable fluorescent dyes facilitates the identification of labeled hydrolytic products as indicators of either diseased or normal tissue where the products of the both hydrolytic reactions (generated using normal and diseased tissue extracts) are combined prior to their separation and detection. In another aspect, quantiative comparison of the amount of each labeled hydrolytic product obtained can be used to determine the relative proportion of affected cells as compared to normal cells in the diseased tissue or, in another aspect, as an indicator of the presence of pre-neoplastic condition or stage of progression of a cancer or tumor.

In some embodiments, there are provided compositions and methods that can be used for the detection of infectious agents, as well as for developing a therapeutic regimen for treatment of that infection and/or development of a prognosis therefor. In one aspect, the infectious agent is the HIV virus. In some embodiments, there are provided compositions comprising one or more multifunctional tags comprising peptide sequences that are specifically or substantially specifically hydrolyzed by one or more particular HIV-specific proteases is contacted with a tissue sample, which is generally a blood sample taken from the individual to be tested. Generation of a labeled hydrolytic product from the HIV-protease-specific multifunctional tag is indicative of the presence of the virus or, more specifically of cells infected by the virus. Moreover, the amount of product can provide an estimate of the viral load, and, accordingly, an indication of the patient's prognosis. In addition, where additional hydrolysis reactions are carried out in the presence of one or more protease inhibitors, it may be apparent which particular protease inhibitor or “cocktail” thereof would be most effective for treatment of that individual patient. Similarly, such methods could also be used to monitor the effectiveness of such treatment as well as the progression of that infection, and to indicate where such treatment is to be adjusted as, or if, drug-resistant variants arise.

3.6.3 Drug Discovery and Analysis

In some embodiments, there are provided compositions and methods that can be used for the discovery of new therapeutic agents, particularly new protease inhibitors. In one aspect of this embodiment a sample containing a single purified, or a substantially-unfractionated, target protease is contacted with a composition comprising a multifunctional tag, the peptide substrate of which is specifically cleaved by the target protease, either in the presence of in the absence of a test compound. The test compound is inferred to be an inhibitor of the target protease where the amount of labeled hydrolytic product detected decreases in the presence thereof. In some embodiments, the products of the hydrolytic reaction are analyzed separately and compared. In some embodiments, hydrolytic reactions carried out using multifunctional tags differing with respect to the reporter, wherein the different reporters are spectrally resolvable fluorescent dyes and/or differing with respect to the mobility modifier attached thereto. In this aspect therefore, the labeled hydrolytic products generated by reactions carried out with and without the test compound can be combined and analyzed together. In some embodiments, a plurality of test compounds, e.g. a population of molecules generated via combinatorial chemical procedures, is tested for the presence of one or more protease inhibitors against the target protease in the sample tested.

In some embodiments, a sample comprising a plurality of purified and/or substantially-unfractionated proteases is contacted with a composition comprising a plurality of different multifunctional tags either in the presence or the absence of the test compound or test compounds. Each different multifunctional tag comprises a different mobility-modifier and a peptide substrate that is specific or substantially specific for one of the proteases in the sample. In this manner, a single test compound is examined for protease-inhibitor activity against a plurality of proteases. In some embodiments, a plurality of test compounds, e.g. a population of molecules generated via combinatorial chemical procedures, is tested for the presence of one or more protease inhibitors against the plurality of proteases. As above, the hydrolytic reactions can be carried in the absence of any test compound using one set of multifunctional tags comprising a first reporter and in the presence of the test compound(s) using a second set of multifunctional tags that comprise a second reporter, wherein the first and second reporters are spectrally resolvable fluorescent dyes. In some embodiments, the population of labeled hydrolytic products generated by the plurality of hydrolytic reactions carried out both with and without the test compound(s) can be combined and analyzed simultaneously.

In some embodiments, there are provided compositions and methods that can be used to evaluate the specificity and/or the potential toxicity of candidate protease inhibitors. Here, for example, a compound identified an inhibitor of a therapeutically-important target protease is tested for its ability to inhibit one or more proteases of the host to which it will be administered. Such reactions can be carried out using one or more purified or substantially-unfractionated host proteases or one or more protease-containing extracts derived from normal tissues of the host and one or a plurality of multifunctional tags of the present invention, each of which comprises a peptide substrate specifically or substantially-specifically hydrolyzed by a protease or protease family of the host. Data indicating that one or more proteases of the host, other than the target protease, are inhibited by the compound of interest would suggest that the compound is potentially toxic to the host.

3.6.4 Basic Research

The proteasome is a large (˜2 MDa) heterocomplex that plays a major role in the degradation of proteins in eukaryotic cells. In this role, the proteasome is directly involved in antigen processing, degradation of misfolded proteins, and turnover of regulatory proteins and transcription factors. Therefore, in some embodiments, there are provided compositions and methods that can be used to facilitate the simultaneous detection and measurement of the proteolytic activity of multiple proteases in a cell, tissue, or other biological system being analyzed by a researcher, including the simultaneous analysis of the various proteolytic activities of the eukaryotic proteasome.

3.6.5 Use of Multifunctional Tags for Detection and/or Quantitation of Hydrolytic Enzymes Other than Proteases

Although the above sections have described the construction and use of multifunctional tags for the analysis of protease activity, in some embodiments, the multifunctional tags disclosed above can be adapted to the multiplexed analysis of other hydrolytic enzymes as well. That is, by replacing the peptide substrate of the multifunctional tags of the present invention with, for example, with an oligosaccharide, a multifunctional tag could be constructed that would enable the detection and quantitation of a particular, catalytically active, endoglycosidase in a sample. Where different, endoglycosidase substrates are identified, a population of different multifunctional tags can be assembled, where each different multifunctional tag comprises a partitioner, a different mobility modifier, and an oligosaccharide that is specifically or substantially specifically hydrolyzed by a particular endoglycosidase or family of endoglycosidases. Again, the mobility modifier will confer a distinctive mobility on corresponding hydrolytic product to which it is attached where the hydrolytic product comprises a reporter and the mobility modifier but does not comprise a partitioner.

In a similar manner, multifunctional tags can be constructed and used for the analysis of enzymes, as well as certain small-molecule antineoplastic agents, with nucleolytic activity. In some embodiments, peptide substrates of the multifunctional tags described above are replaced with a nucleic acid, which can comprise a single-stranded oligonucleotide or a double-stranded DNA molecule. In some embodiments, the mobility modifier, e.g. comprising a reporter, can be attached to one end of a first oligodeoxyribonucleotide while a partitioner is attached to the other end of the oligodeoxyribonucleotide. A second oligodeoxyribonucleotide, comprising a nucleic acid sequence complementary to the first oligodeoxyribonucleotide, is annealed to provide a double-stranded DNA molecule comprising the nucleotide sequence recognized and hydrolyzed by a restriction endonuclease. Where a particular restriction endonuclease is characteristic of a pathogenic microorganism, then such a multifunctional tag could be used e.g. as a diagnostic reagent for the presence of the pathogen. In some embodiments, such a multifunctional tag, comprising a duplex DNA molecule as a substrate, could be used for the detection of molecules that bind to DNA and lead scission of one or both strands. Such molecules include, but are not limited to, those of the enediyne family of compounds which encompasses the following three groups of molecules: (1) the calichaemyicin-esperamicin type compounds, (2) the dynemicin type compounds, and (3) the chromoprotein type compounds.

### 4. EXAMPLE

### Multiplexed Detection of Proteases

Five different proteases (the serine protease, prostate-specific antigen; the matrix metalloprotease, matrilysin; HIV-1 protease; plasmin; and tissue plasminogen activator) are detected and quantitated, according to one embodiment of the invention.

4.1 Synthesis of the Partitioner

For purposes of the present illustration, the partitioner is a cationic block copolymer comprising a sufficient number of positively charge moieties such that the multifunctional tag, as a whole is positively charged. This will facilitate, e.g. an electrophoretic separation of nonhydrolyzed multifunctional tags from the negatively-charged labeled hydrolytic products comprising an anionic mobility modifier and the reporter.

In this illustration, the cationic block has the structure H[NH(CH₂)₃NH(CH₂)₄NH(CH₂)₃]ₓNH₂, where x has a value within the range of at least 5 to about 15, preferably for this illustration, x is about 10. Such a cationic block is synthesized and isolated, for example, according to the methods disclosed in U.S. Pat. No. 6,221,959 B1, which is hereby incorporated by reference in its entirety. However, with respect to the following steps, the product fractions retained include those products having a nominal molecular weight of 2000.

One equivalent of H[NH(CH₂)₃NH(CH₂)₄NH(CH₂)₃]ₓ—NH₂, where x is about 10, is condensed with one equivalent of the methyl ether of polyethylene glycol-succinimidyl propionate (mPEG-SPA (MW 2000) Shearwater Corporation (Huntsville, Ala.). The desired intermediate formed by condensation of one molecule of MPEG-SPA with one molecule of H[NH(CH₂)₃NH(CH₂)₄NH(CH₂)₃]ₓ—NH₂, where x is about 10, to provide intermediates having an average molecular weight of approximately 5000, which have one free, primary amino group.

One equivalent of isolated intermediate having a structure that can be represented as mPEG-C(O)NH[(CH₂)₃NH(CH₂)₄NH(CH₂)₃NH]ₓH, where x is about 10, is then condensed with one equivalent of (FMOC)NH(CH₂CH₂O)_{y}CH₂CH₂CO₂NHS, having a nominal molecular weight of 3400 (where y is approximately 75), available from Shearwater Corporation (Huntsville, Ala.) to provide a cationic block copolymer consisting of one cationic block that is bracketed by two polyethylene glycol blocks (one of which is blocked with a methyl-ether moiety, while the other comprises an FMOC-protected primary amino group), and having a nominal molecular weight of approximately 8400 and carrying a net charge at neutral pH of approximately +30. Removal of the FMOC group under standard conditions provides a cationic block copolymer with a primary amine group that can be condensed, using standard methods and reagents well known in the art with, for example, an activated derivative, such as and N-hydroxysuccinimidyl (NHS) ester of a carboxylic acid.

4.2 Synthesis of the Peptide Substrates

Phage display analyses have been used to identify peptide substrates that effectively cleaved by each of the proteases be detected and therefore are used in the construction of the peptide substrate of multifunctional tags that are substantially specific for each of following: (1) SSFYSS, which is cleaved between the fourth (Y) and fifth (S) amino acid by the serine protease prostate-specific antigen (PSA) (see e.g. Coombs et al. (1998) Chemistry and Biology 5: 475-88), (2) PLELRA which is cleaved between the third (E) and fourth (L) amino acid by matrilysin (see e.g. Smith et al. (1995) J. Biol. Chem. 270(12): 6440-49), (3) GSGIFLETSL which is cleaved between the fifth (F) and sixth (L) amino acid by HIV-1 protease (see e.g. Beck et al. (2000) Virology 274: 391-401), (4) LGGSGIYRSRSLE which is cleaved between the eighth (R) and ninth (S) amino acid by plasmin (see e.g. Hervio et al. (2000) Chemistry and Biology 7: 443-53), and (5) GGSGPFGRSALVPE which is cleaved between the eighth (R) and ninth (S) amino acid by tissue-type plasminogen activator (see e.g. Ding et al. (1995) Proc. Natl. Acad. Sci. 92: 7627-31).

In each instance a peptide to be synthesized is bracketed at both the amino-terminal and carboxy-terminal ends with a five-amino acid sequence (GGPGG) to provide flexibility to the peptide substrate and to disrupt structural influences, if any, provided by the attached partitioner and mobility modifier. Accordingly the following five peptides (described using single-letter codes) are synthesized with standard solid phase methods (see e.g. Fields and Noble (1990) Int. J. Peptide Protein Res. 35: 161-214) preferably employing an automated instrument such as, but not limited to the Pioneer™ Peptide Synthesizer or the ABI 433A Peptide Synthesizer (Applied Biosystems, Foster City, Calif.), employing commercially-available reagents well known in the art:

1. GGPGGSSFYSSGGPGG
2. GGPGGPLELRAGGPGG
3. GGPGGGSGIFLETSLGGPGG
4. GGPGGLGGSGIYRSRSLEGGPGG
5. GGPGGGGSGPFGRSALVPEGGPGG

4.3 Synthesis and Attachment of the Mobility Modifiers

In each instance in the present illustration, the mobility modifier is added to the amino terminus of each of the resin bound peptides, to provide a mobility-modified peptide substrate having a net charge of −4 at neutral pH. The mobility modifier attached to the first peptide substrate is made up of 6 ethylene oxide monomeric units wherein 4 of the linkages are formed using a phosphodiester linkage; the mobility modifier attached to the second peptide substrate is made up of 10 ethylene oxide monomeric units wherein 3 of the linkages are formed using a phosphodiester linkage; the mobility modifier attached to the third peptide substrate is made up of 14 ethylene oxide monomeric units wherein 7 of the linkages are formed using a phosphodiester linkage; the mobility modifier attached to the fourth peptide substrate is made up of 18 ethylene oxide monomeric units wherein 6 of the of the linkages are formed using a phosphodiester linkage; and the mobility modifier attached to the fifth peptide substrate is made up of 22 ethylene oxide monomeric units wherein 5 of the linkages are formed using a phosphodiester linkage. In each instance, the last monomer unit added comprises a free amino group that is not involved in the linkage formed with the penultimate monomer unit.

4.4 Attachment of the Reporter

The N-hydroxysuccinamide (NHS) derivatives of fluorescent dyes that can be directly coupled to the primary amine of the mobility modified peptide substrates include, but are not limited to, JOE (6-carboxy-4′,5′-dichloro-2′,7′-dimethoxyfluorescein), ROX™ (carboxy-X-rhodamine), and TAMRA™ (carboxytetramethylrhodamine), which are well known in the art and are commercially available from, e.g., Integrated DNA Technologies, (Coralville, Iowa), or NHS esters of the water soluble rhodamine dyes disclosed in U.S. Pat. No. 6,372,907 B1, which discloses methods for covalently attaching such dyes to a primary amino group, and which is hereby incorporated by reference in it entirety. For the purposes of the present illustration, the reporter to be attached to the terminal, primary amino group of the mobility modified attached to the peptide substrate is TAMRA™ (carboxytetramethylrhodamine). Accordingly, the NHS ester of TAMRA™ (carboxytetramethylrhodamine) is condensed with the terminal amino group of each of the mobility modified peptide substrates yielding a peptide bond as a linkage group, to provide the following compounds, after removal from the solid substrate and deprotection:

1. TAMRA ™-(EO)₆-GGPGGSSFYSSGGPGG-COOH
2. TAMRA ™-(EO)₁₀-GGPGGPLELRAGGPGG-COOH
3. TAMRA ™-(EO)₁₄-GGPGGGSGIFLETSLGGPGG-COOH
4. TAMRA ™-(EO)₁₈-GGPGGLGGSGIYRSRSLEGGPGG-COOH,
and
5. TAMRA ™-(EO)₂₂-GGPGGGGSGPFGRSALVPEGGPGG-COOH.

4.5 Attachment of the Partitioner

As noted above the cationic partitioner includes a free, primary amino group. Accordingly, the terminal carboxy group of each of the five different, mobility modified peptide substrates of the previous section, is activated, e.g. with N-hydroxysuccinamde, and then condensed with the primary amino group of the partitioner, under standard conditions well known in the art. The desired product of each of the five reactions is a unique multifunctional tag comprising a cationic partitioner, peptide substrate, mobility moidifier and reporter molecule.

4.6 Hydrolysis of the Multifunctional Tags

Aliquots of each of the five multifunctional tags assembled in sections 6.1-6.6, supra are combined and contacted with a solution of comprising the serine protease prostate-specific antigen, the matrix metalloprotease matrilysin, HIV-1 protease, plasmin, and tissue plasminogen activator and incubated at a suitable temperature and for a time sufficient to allow hydrolysis of each multifunctional tag by the cognate protease.

4.7 Separation, Detection, and Analysis of the Labeled Hydrolysis Products

The mixture of hydrolyzed multifunctional tags generated in section 6.6 is then analyzed by non-sieving capillary electrophoresis (CE). The methods and apparatus used to carry out the CE separations according to the present invention are performed using conventional CE methods and apparatus, as generally described elsewhere (e.g., Capillary Electrophoresis Theory and Practice, Grossman and Colburn, eds., Academic Press (1992)). Standard polyimide-coated fused silica capillary tubes, fluid separation medium, i.e. a buffered polymer solution or, in the alternative, a polymer-free buffer solution, sample injection techniques, i.e. electrokinetic injection, an automated system control devices, including a digital computer and automated fluorescence detection equipment are used. More specifically, the electrophoretic separation is carried out using an ABI PRISM™ 3700 DNA Analyzer (PE Biosystems, p/n 4308058, Foster City, Calif.) equipped with a 50 cm capillary array (p/n 4305787). The 3700 system includes a plurality of individual, fused-silica separation capillaries, each capillary having an uncoated interior surface, a total length of 50 cm, an effective separation length of 50 cm, and in internal diameter of 50 μm. Fluorescence detection of the sample analytes in the 3700 system is accomplished using a sheath-flow detection system (e.g., as described in Kambara et al., U.S. Pat. No. 5,529,679; and Dovichi et al., U.S. Pat. No. 5,439,578). Samples are electrokinetically injected into the capillaries by applying an electric field of 50 V/cm for 30 seconds while the inlet end of the capillary is immersed in the sample mixture. The separation medium used comprises 75 mM tris-phosphate, pH 7.6 and the commercially-available ABI PRISM™ 3700 POP6 polymer (p/n 4306733, PE Biosystems, Foster City, Calif.), which is a solution of a linear substituted polyacrylamide. Fluorescence is induced by excitation with a 40 mW Ar ion laser. The grounded cathodic reservoir and the anodic reservoirs are filled with a buffer comprising 75 mM tris-phosphate, pH 7.6. About 2 nanoliters of solution are drawn into the cathodic end of the tube by electrokinetic injection. The electrophoretic system is run at a voltage setting of about 15 kV (about 270 V/cm) throughout the run. Fluorescence detection is at 530 nm. The detector output signal corresponding to each hydrolytic product is integrated and plotted using the software provided with the ABI PRISM™ 3700.

1. A multifunctional tag composition for use in detecting the presence or absence of one or more catalytically-active target proteases in a sample, the composition comprising a plurality of different multifunctional tags, wherein each different multifunctional tag comprises
(a) a peptide substrate that is substantially specifically hydrolyzable by a different catalytically-active target protease;
(b) a distinctive mobility modifier attached to the peptide substrate;
(c) a partitioner attached to the peptide substrate; and
(d) a reporter;
wherein hydrolysis of the peptide substrate of each different multifunctional tag by a different catalytically-active target protease provides a different labeled hydrolytic product, wherein each different labeled hydrolytic product comprises a reporter and a distinctive mobility modifier but does not comprise a partitioner, and wherein each distinctive mobility modifier imparts to each different labeled hydrolytic product an electrophoretic mobility that is distinctive relative to the electrophoretic mobility of other different multifunctional tags in said composition and of other labeled hydrolytic products produced by hydrolysis of the peptide substrate of different multifunctional tags.

2. The multifunctional tag composition of claim 1, wherein each reporter is attached to a peptide substrate.

3. The multifunctional tag composition of claim 1, wherein each reporter is attached to a mobility modifier.

4. The multifunctional tag composition of claim 1, wherein each peptide substrate comprises fewer than 50 amino acids.

5. The multifunctional tag composition of claim 4, wherein each peptide substrate comprises fewer than 40 amino acids.

6. The multifunctional tag composition of claim 5, wherein each peptide substrate comprises fewer than 30 amino acids.

7. The multifunctional tag composition of claim 6, wherein each peptide substrate comprises fewer than 20 amino acids.

8. The multifunctional tag composition of claim 7, wherein each peptide substrate comprises fewer than 15 amino acids.

9. The multifunctional tag composition of claim 1, wherein each mobility modifier is a substantially monodisperse polymer.

10. The multifunctional tag composition of claim of claim 9, wherein said polymer is selected from the group consisting of polyethylene oxide, polyglycolic acid, polylactic acid, oligosaccharide, polyurethane, polyamide, polyamine, polyimine, polysulfonamide, polysulfoxide, and block copolymers thereof.

11. The multifunctional tag composition of claim 9, wherein said polymer comprises a polyethylene oxide polymer.

12. The multifunctional tag composition of claim 11, wherein said polymer comprises a charged linking group.

13. The multifunctional tag composition of claim 12, wherein the charged linking group is a phosphodiester linking group.

14. The multifunctional tag composition of claim 11, wherein said polymer comprises an uncharged linking group.

15. The multifunctional tag composition of claim 14, wherein the uncharged linking group is a phosphotriester linking group.

16. The multifunctional tag composition of claim 1, wherein at least one different multifunctional tag comprises a mobility-modifier, wherein the mobility-modifier is non-covalently attached to the peptide substrate.

17. The multifunctional tag composition of claim 16, wherein at least one different multifunctional tag comprises a peptide substrate comprising a first nucleic acid and wherein the composition further comprises a mobility modifier comprising a second nucleic acid, wherein the first and second nucleic acids are complementary.

18. The multifunctional tag composition of claim 17, wherein at least one of the first and the second nucleic acids comprises a peptide nucleic acid.

19. The multifunctional tag composition of claim 1, wherein each partitioner comprises a polymer.

20. The multifunctional tag composition of claim 19, wherein said polymer comprises at least one of polyethylene oxide, polyglycolic acid, polylactic acid, oligosaccharide, polyurethane, polyamide, polyamine, polyimine, polysulfonamide, polysulfoxide, or a block copolymer thereof.

21. The multifunctional tag composition of claim 19, wherein said polymer comprises a polyethylene oxide polymer.

22. The multifunctional tag composition of claim 21, wherein said polymer comprises a charged linking group.

23. The multifunctional tag composition of claim 22 wherein the charged linking group is a phosphodiester linking group.

24. The multifunctional tag composition of claim 21, wherein said polymer comprises an uncharged linking group.

25. The multifunctional tag of claim 24, wherein the uncharged linking group is a phosphotriester linking group.

26. The multifunctional tag composition of claim 19, wherein said polymer is a substantially monodisperse polymer.

27. The multifunctional tag composition of claim 1, wherein each different multifunctional tag has a net negative electrostatic charge, wherein each partitioner has a net negative electrostatic charge, and wherein each labeled hydrolytic product has a net positive electrostatic charge.

28. The multifunctional tag composition of claim 1, wherein each different multifunctional tag has a net positive electrostatic charge, wherein each partitioner has a net positive electrostatic charge, and wherein each labeled hydrolytic product has a net negative electrostatic charge.

29. The multifunctional tag composition of claim 1, wherein the molecular weight of each partitioner is at least twice the molecular weight of each labeled hydrolytic product.

30. The multifunctional tag composition of claim 29, wherein the molecular weight of each partitioner is at least five-fold greater than the molecular weight of each labeled hydrolytic product.

31. The multifunctional tag composition of claim 30, wherein the molecular weight of each partitioner is at least ten-fold greater than the molecular weight of each labeled hydrolytic product.

32. The multifunctional tag composition of claim 1, wherein each reporter comprises a fluorescent dye.

33. The multifunctional tag composition of claim 1, wherein at least one first different multifunctional tag comprises a first reporter and at least one second different multifunctional tag comprises a second reporter, wherein the first and second reporters are different.

34. The multifunctional tag composition of claim 33, wherein the first and second reporters comprise spectrally-resolvable fluorescent dyes.

35. A method for detecting the presence or absence of one or more catalytically-active target proteases in a sample, the method comprising
a) contacting the sample with a multifunctional tag composition under selected hydrolysis conditions to provide a reaction mixture, wherein the multifunctional tag composition comprises a plurality of different multifunctional tags, wherein each different multifunctional tag comprises (i) a peptide substrate that is substantially specifically hydrolyzed by a different catalytically-active protease, (ii) a distinctive mobility modifier attached to each peptide substrate, (iii) a partitioner attached to each different peptide substrate, and (iv) a reporter, wherein hydrolysis of each different multifunctional tag by a different target protease provides a different labeled hydrolytic product, wherein each different labeled hydrolytic product comprises a distinctive mobility modifier and a reporter but does not comprise a partitioner, and wherein the mobility modifier imparts to each different labeled hydrolytic product an electrophoretic mobility that is distinctive relative to the electrophoretic mobility of the other different multifunctional tags in the composition and of other different labeled hydrolytic products in the reaction mixture;
b) fractionating the reaction mixture using a mobility-dependent analysis technique; and
c) detecting one or more different labeled hydrolytic products, wherein the presence of each different labeled hydrolytic product indicates that a different catalytically-active target protease is present in the sample.

36. The method of claim 35, wherein the amount of each different labeled hydrolytic product is substantially proportional to the amount of each different catalytically-active target protease present in the sample.

37. The method of claim 36, wherein the mobility-dependent analysis technique comprises electrophoresis.

38. The method of claim 37, wherein the mobility-dependent analysis technique comprises electrophoresis in a sieving medium.

39. The method of claim 37, wherein the mobility-dependent analysis technique comprises electrophoresis in a non-sieving medium.

40. The method of claim 35, wherein said fractionating is carried out using capillary electrophoresis.

41. The method of claim 40, wherein the capillary electrophoresis is carried out in the presence of an affinophore comprising a first ligand, and at least one multifunctional tag of the composition comprises a mobility modifier comprising a second ligand, wherein the first ligand and the second ligand are members of a binding pair.

42. The method of claim 41, wherein the first ligand comprises a first nucleic acid and the second ligand comprises a second nucleic acid, wherein the first nucleic acid is complementary to the second nucleic acid.

43. The method of claim 42, wherein at least one of the first and second nucleic acids is a peptide nucleic acid.

44. A kit for detecting the presence or absence of one or more catalytically-active target proteases in a sample, the kit comprising in one or more containers an amount of a plurality of different multifunctional tags, wherein each different multifunctional tag comprises
(a) a peptide substrate substantially specifically hydrolyzed by a different catalytically-active target protease;
(b) a distinctive mobility modifier attached to the peptide substrate;
(c) a partitioner attached to the peptide substrate; and
(d) a reporter;
wherein hydrolysis of the peptide substrate of a different multifunctional tag by a different catalytically-active target protease provides a different labeled hydrolytic product, wherein each different labeled hydrolytic product comprises a reporter and a distinctive mobility modifier but does not comprise a partitioner, and wherein each distinctive mobility modifier imparts to each different labeled hydrolytic product an electrophoretic mobility that is distinctive relative to the electrophoretic mobility of the other different multifunctional tags and of other different labeled hydrolytic products provided by hydrolysis of the peptide substrate of different multifunctional tags by different catalytically-active target proteases.

45. A method for diagnosing a disease in a subject comprising
(a) providing a sample derived from a tissue of the subject, wherein the sample comprises at least one catalytically-active target protease;
(b) providing a multifunctional tag composition comprising a plurality of different multifunctional tags, wherein each different multifunctional tag comprises (i) a peptide substrate substantially specifically hydrolyzed by a different catalytically-active target protease, (ii) a distinctive mobility modifier attached to the peptide substrate, (iii) a partitioner attached to the peptide substrate, and (iv) a reporter;
(c) contacting the sample and the multifunctional tag composition under selected hydrolysis conditions to provide a reaction mixture, wherein hydrolysis of each different multifunctional tag by each different catalytically-active target protease provides a different labeled hydrolytic product, wherein each different labeled hydrolytic product comprises a distinctive mobility modifier and a reporter but does not comprise a partitioner, and wherein the mobility modifier imparts to each different labeled hydrolytic product an electrophoretic mobility that is distinctive relative to the electrophoretic mobility of the other different multifunctional tags in the reaction and of other different labeled hydrolytic products in the reaction, and wherein a first labeled hydrolytic product is diagnostic of normal tissue and a second labeled hydrolytic product is diagnostic of diseased tissue;
d) fractionating the reaction mixture using a mobility-dependent analysis technique; and
e) detecting each different labeled hydrolytic product, wherein the presence of a greater amount of the first labeled hydrolytic product as compared to the amount of the second labeled hydrolytic product indicates that the tissue is normal, and wherein the presence of a greater amount of the second labeled hydrolytic product as compared to the amount of the first labeled hydrolytic product indicates that the tissue is diseased.

45. The method of claim 44, wherein the diseased tissue is tissue of a type of cancer.

47. The method of claim 45, wherein the diseased tissue is infected with an infectious agent selected from the group consisting of bacterial, fungal, parasitic, and viral infectious agents.

48. The method of claim 47 wherein the infectious agent is a viral infectious agent.

49. The method of claim 48, wherein the viral infectious agent is an HIV virus.

50. The method of claim 48, wherein the viral infectious agent is a causative agent of SARS.

51. A method of screening for therapeutic agents useful for the prevention and treatment of disease comprising
(a) providing a sample comprising a plurality of different catalytically-active target proteases, wherein each different target protease is diagnostic of a different target disease;
(b) providing a first multifunctional tag composition comprising first set of first different multifunctional tags, wherein each first multifunctional tag comprises (i) a first peptide substrate substantially specifically hydrolyzed by a different catalytically-active target protease, (ii) a first distinctive mobility modifier attached to the first peptide substrate, (iii) a first partitioner attached to the first peptide substrate, and (iv) a first reporter;
(c) providing a second composition comprising a test compound and a second set of second different multifunctional tags, wherein each second different multifunctional tag comprises (i) a second peptide substrate substantially specifically hydrolyzed by a different target protease, (ii) a second distinctive mobility modifier attached to the second peptide substrate, (iii) a second partitioner attached to the second peptide substrate, and (iv) a second reporter;
(d) contacting an aliquot of the sample and the first multifunctional tag composition under selected hydrolysis conditions to provide a first reaction mixture and to provide a first set of different labeled hydrolytic products, wherein each first different labeled hydrolytic product comprises a first distinctive mobility modifier and a first reporter but not a first partitioner, whereby each first different labeled hydrolytic product has a ratio of charge/translational frictional drag that is distinctive relative to the charge/translational frictional drag ratios of the first and second different multifunctional tags and relative to the charge/translational frictional drag ratios of other first different labeled hydrolytic products in the first reaction mixture, and wherein the amount of each first different labeled hydrolytic product is proportional to the total catalytic activity of a different catalytically-active target protease in the absence of a test compound;
(e) contacting an aliquot of the sample and the second multifunctional tag composition under selected hydrolysis conditions to provide a second reaction mixture and to provide a second set of second different labeled hydrolytic products, wherein each second different labeled hydrolytic product comprises a second distinctive mobility modifier and a second reporter but not a second partitioner whereby each second different labeled hydrolytic product has an electrophoretic mobility that is distinctive relative to the electrophoretic mobility of the first and of the second different multifunctional tags and is distinctive relative to the electrophoretic mobility of the first different labeled hydrolytic products and other second different labeled hydrolytic products in the second reaction mixture, and wherein the amount of each second labeled hydrolytic product is proportional to the total catalytic activity of a different catalytically-active target protease in the presence of the test compound;
f) combining the first and second reaction mixtures to provide a combined reaction mixture;
g) fractionating the combined reaction mixture using a mobility-dependent analysis technique;
h) detecting each first different labeled hydrolytic product and each second different labeled hydrolytic product; and
e) comparing the amount of first different labeled hydrolytic product provided by hydrolysis of the peptide substrate of a first different multifunctional tag by a specific catalytically-active target protease and the amount of second different labeled hydrolytic product provided by hydrolysis of the peptide substrate of a second different multifunctional tag by the specific catalytically-active target protease.

52. The method of claim 51, wherein each first partitioner and each second partitioner are the same.

53. The method of claim 51, wherein a first peptide substrate and a second peptide substrate are the same.

54. The method of claim 51, wherein a first different multifunctional tag comprises a first peptide substrate, a first mobility modifier and a first reporter, wherein a second different multifunctional tag comprises a second peptide substrate, a second mobility modifier and a second reporter, wherein the first peptide substrate and the second peptide substrate are the same, wherein the first mobility modifier and the second mobility modifier are the same, and wherein first reporter is a first fluorescent dye and the second reporter is a second fluorescent dye, wherein the first and second fluorescent dyes are spectrally-resolvable fluorescent dyes.

55. The method of claim 51, wherein a first different multifunctional tag comprises a first peptide substrate, a first mobility modifier and a first reporter, wherein a second different multifunctional tag comprises a second peptide substrate, a second mobility modifier and a second reporter, wherein the first and second peptide substrates are the same, wherein the first and second reporters are the same, wherein hydrolysis of the first different multifunctional tags by a target protease provides a first labeled hydrolytic product comprising a first mobility modifier, and hydrolysis of the second different multifunctional tag by the target protease provides a second different hydrolytic product comprising the second mobility modifier, wherein the first mobility modifier imparts an electrophoretic mobility to the first labeled hydrolytic product that is distinctive relative to the electrophoretic mobility imparted by the second mobility modifier to the second different labeled hydrolytic product.


SEQUENCE LISTING (14 sequences)

SEQ ID NO: 1 - protein, 8 aa, Unknown
  general structure of amino acid peptide hydrolyzed by a protease (including prime-side and non-prome side amino acids)
XXXXXXXX

SEQ ID NO: 2 - protein, 4 aa, Unknown
  spacer or linking peptide
GPGG

SEQ ID NO: 3 - protein, 4 aa, Unknown
  spacer or linking peptide
GGPG

SEQ ID NO: 4 - protein, 6 aa, Unknown
  peptide recognized and cleaved by serine protease prostate-specific antigen (PSA)
SSFYSS

SEQ ID NO: 5 - protein, 6 aa, Unknown
  peptide recognized and cleaved by matrilysin
PLELRA

SEQ ID NO: 6 - protein, 10 aa, Unknown
  peptide recognized and cleaved by HIV-1 protease
GSGIFLETSL

SEQ ID NO: 7 - protein, 13 aa, Unknown
  peptide recognized and cleaved by plasmin
LGGSGIYRSRSLE

SEQ ID NO: 8 - protein, 14 aa, Unknown
  peptide recognized and cleaved by tissue-type plasminogen activator
GGSGPFGRSALVPE

SEQ ID NO: 9 - protein, 5 aa, Artificial Sequence
  sequence to provide flexibility to the peptide substrate and to disrupt structural influences by adding to both ends of the peptide to be synthesized
GGPGG

SEQ ID NO: 10 - protein, 16 aa, Artificial Sequence
  Synthetic peptide using partitioner and mobility modifier
GGPGGSSFYSSGGPGG

SEQ ID NO: 11 - protein, 16 aa, Artificial Sequence
  Synthetic peptide using partitioner and mobility modifier
GGPGGPLELRAGGPGG

SEQ ID NO: 12 - protein, 20 aa, Artificial Sequence
  Synthetic peptide using partitioner and mobility modifier
GGPGGGSGIFLETSLGGPGG

SEQ ID NO: 13 - protein, 23 aa, Artificial Sequence
  Synthetic peptide using partitioner and mobility modifier
GGPGGLGGSGIYRSRSLEGGPGG

SEQ ID NO: 14 - protein, 24 aa, Artificial Sequence
  Synthetic peptide using partitioner and mobility modifier
GGPGGGGSGPFGRSALVPEGGPGG